(19) United States
(12) Patent Application Publication
(10) Pub. No.: US 2026/0325801 A1
(43) Pub. Date: Oct. 22, 2026

(54) A CATHETER FOR HYDRODYNAMIC INJECTION

(71) Applicant: HYDROGENE THERAPEUTICS, INC., Houston, TX (US)

(72) Inventor: Robert KRUSE, Houston, TX (US)

(73) Assignee: HYDROGENE THERAPEUTICS, INC., Houston, TX (US)

(21) Appl. No.: 19/107,563

(22) PCT Filed: Aug. 31, 2023
(86) PCT No.: PCT/US2023/031726

§ 371 Date: Feb. 27, 2025

Related U.S. Application Data
(60) Provisional application No. 63/374,237, filed on Aug. 31, 2022.

(51) Int. Cl. A61M 25/10; A61F 2/95; A61F 2/962; A61M 25/00

(52) U.S. Cl. CPC A61M 25/1002; A61F 2/962; A61M 25/0023; A61M 25/0054; A61F 2002/9528; A61F 2210/0014; A61F 2230/0008; A61M 2025/0001; A61M 2025/1079; A61M 2025/1093; A61M 2202/0007; A61M 2202/04; A61M 2205/0238; A61M 2205/0266; A61M 2205/32; A61M 2205/3327; A61M 2205/3334; A61M 2205/3344; A61M 2205/50; A61M 2210/1039; A61M 2210/1075; A61M 2210/1082; A61M 2210/1085; A61M 2210/1089; A61M 2210/12

(57) ABSTRACT

The present disclosure relates to an apparatus and system for hydrodynamic injection for gene therapy. More particularly, the present disclosure relates to an apparatus and system for hydrodynamic injection of gene therapy therapeutic agents into large animals and humans.

### CROSS-REFERENCE TO RELATED APPLICATIONS

The present application is related to and claims priority under 35 U.S.C. § 119(e) to U.S. Provisional Patent Application No. 63/374,237, entitled “A CATHETER FOR HYDRODYNAMIC INJECTION,” filed Aug. 31, 2022. The entire contents of the aforementioned patent application is incorporated herein by this reference.

### FIELD

The present disclosure relates to an apparatus and system for hydrodynamic injection for gene therapy. More particularly, the present disclosure relates to an apparatus and system for hydrodynamic injection of gene therapy therapeutic agents into large animals and humans.

### BACKGROUND

Gene therapy is a therapeutic approach designed to treat genetic disease. Gene therapy generally involves introducing a gene therapy agent (e.g., a recombinant nucleic acid) that includes transferred genetic material that is transcribed and/or translated in a patient's cells, tissues, or organs to compensate for or suppress/rescue one or more phenotypes associated with a genetic mutation. For example, an organism that is homozygous for a recessive loss-of-function mutation may be injected with wildtype copies of the affected gene to provide transcription and/or subsequent translation to produce wildtype protein molecules to rescue the loss-of-function homozygous phenotype, thereby treating a genetic disorder associated with the gene mutation.

Gene therapy is generally based on the use of viral gene therapy vectors such as adeno-associated virus or lentivirus. Disadvantageously, such viral gene therapy vectors are constrained with respect to the size of the gene therapy construct (e.g., a nucleotide sequence) that they can package. Another disadvantage of such viral gene therapy vectors is the potential to integrate into the host genome and/or generate a host immune response. Yet another disadvantage of such viral gene therapy vectors is that they are complex to make, test, and manufacture. Accordingly, there is an urgent need for improved systems for delivering a gene therapy agent to a patient.

### SUMMARY

The present disclosure relates to an apparatus and system for hydrodynamic injection of gene therapy agents. More particularly, the present disclosure relates to an apparatus and system for hydrodynamic injection of gene therapy agents into large animals and humans. As described in detail below, the present disclosure is based, at least in part, on the surprising discovery that a hydrodynamic catheter system having specific lumen configurations may be used to deliver gene therapy agents (e.g., one or more recombinant nucleic acids and/or one or more proteins) to a patient.

In an aspect, the disclosure provides a gene delivery catheter system for performing endoscopic-mediated gene and protein delivery into liver, pancreas, and kidney organs, comprising a multi-lumen catheter having a catheter body having a substantially cylindrical shape and a substantially uniform in diameter along its longitudinal axis, and having three or four mutually independent lumens extending through the catheter body, wherein the multi-lumen catheter for insertion into a working channel of an endoscope is formed and dimensioned, characterized in that—the multi-lumen catheter contains:
(a) an optional first lumen for receiving a pressure catheter/transducer,
(b) a second lumen for receiving a guidewire,
(c) a third lumen for receiving and transmitting a solution, and
(d) a fourth lumen for transmitting air to inflate a balloon at the distal end of the catheter

In exemplary embodiments, the second lumen and the third lumen can be combined into a single lumen accomplishing both tasks, with the guidewire removed prior to nucleic acid or protein solution injection.

In exemplary embodiments, the guidewire and solution injection lumen are the two largest lumens compared to the pressure catheter and air-balloon lumens.

In exemplary embodiments, the third solution lumen is optimally the largest lumen in diameter for injection of nucleic acid and/or protein, and the other three lumens are minimal in size for their functional purpose.

In exemplary embodiments, the largest lumen diameter used for hydrodynamic injection of nucleic acid and/or protein solution will allow for decreased catheter wall tension during injection and greater flow rates tolerated.

In exemplary embodiments, the first lumen of the multi-lumen catheter for the pressure sensor has a diameter of up to 300 microns, 400 microns, 500 microns, 600 microns, 700 microns, or 800 microns to fit commercial pressure sensor sizes.

In exemplary embodiments, the pressure sensor inserted into the multi-lumen catheter system is connected a pressure transducer, such that real-time pressure can be monitored during the injection

In exemplary embodiments, the pressure sensor inserted into the multi-lumen catheter system is connected to external electronic system, such that real-time pressure can be monitored during the injection.

In exemplary embodiments, the first or second lumens may an oval or round diameter to facilitate insertion of a substantial circular pressure sensor and guidewire.

In exemplary embodiments, the second lumen of the multi-lumen catheter receiving the guidewire has a diameter just larger than 0.018 inches, 0.025 inches, 0.035 inches, or 0.045 inches to accommodate these different guidewire sizes for insertion through the lumen.

In exemplary embodiments, the first lumen containing the pressure sensor, already has the pressure sensor inserted and/or embedded in it, such that a separate pressure sensor is not required to be manually insert into the multi-lumen catheter system prior to the procedure.

In exemplary embodiment, a five-lumen catheter is available for specific scenarios where leakage monitoring during procedures is necessary. Precise injection is facilitated through strategically positioned injection ports, located both above and, if required, below the adjustable balloon. This configuration allows controlled contrast and DNA injections, tailored to procedural demands.

In exemplary embodiments, the pressure sensor is able to slide and be advanced forward beyond the distal tip of the catheter into the lumen of the duct or vessel in order to sense fluid pressure directly in the lumen during injection.

In exemplary embodiments, the pressure sensor, either inserted or already embedded in the catheter, can be retracted inside the catheter to positions up to 50 cm away from the tip of the catheter, but still sense the correct fluid pressure.

In exemplary embodiments, the pressure sensor can have a length of 195 cm to reach the tip of the catheter.

In exemplary embodiments, the pressure sensor has a length of 140 cm and sit inside the catheter, but still pressure accurate readings.

In exemplary embodiments, the lumen for the air has the smallest diameter among the four lumens and terminates at the proximal end to the user at a connecting end, affording access by syringe for air injection.

In exemplary embodiments, the lumen can preferably have non-circular or non-oval shape in order to accommodate the cross-sectional areas of the other three lumens.

In exemplary embodiments, the lumen inflates a balloon near the distal end of the catheter, wherein the said balloon would be located optimally 0.5 cm, 1 cm, 2 cm, or 3 cm from the end of the catheter.

In exemplary embodiments, shorter distances less than 1 cm assure increased likelihood of not missing any vessel or duct branches during injection.

In exemplary embodiments, the air balloon has a maximal inflation diameter of 8 mm, 10 mm, 15 mm, 20 mm, or 25 mm.

In exemplary embodiments, catheter possesses increased tensile strength of avoid breaks or tears to tolerate flow rates between 2-20 mL/sec and to withstand pressure limits between 500-2000 psi.

In exemplary embodiments, the first three lumens have forward facing exits at the distal end of the catheter.

In exemplary embodiments, the guidewire and injection lumens are forward facing, but the pressure sensor lumen lateral facing lumen just distal to the balloon to avoid detecting fluid turbulence during injection.

In exemplary embodiments, the distal tip of the catheter is tapered with a smaller diameter than the diameter of the major length of the catheter, thereby facilitating insertion into orifices.

In exemplary embodiments, the diameter of the taper is still greater than the diameter of the guidewire, injection lumen, and/or pressure catheter combined, allowing the guidewire and pressure catheter to still exit forward through the catheter.

In exemplary embodiments, the distal tip of the catheter is not tapered and ends blunt with all lumens forward facing in consistent size.

In exemplary embodiment, the distal tip has enhanced flexibility, due to reduction in caliber of the catheter in the distal quarter or third, or use of different materials, enabling maneuverability into the ductal system over a guidewire.

In exemplary embodiments, the total diameter of the catheter is 4.5 French, 5 French, 6 French, 7 French, 8 French, 9 French, or 10 French in size.

In exemplary embodiments, all lumens also contain connectors at their distal ends to allow for connecting of other lines/devices for the injection of fluid through them, if necessary.

In exemplary embodiments, the distal tip of the catheter is steerable with a tension wire running up to but just short of the balloon such that it responds to the opening and closing of a clamp by the user at the proximal end of the catheter.

In exemplary embodiments, the injection port has a lateral opening at a position upstream of the air balloon with respect to the catheter, affording the flow of the injection in the proximal direction after balloon seal.

In exemplary embodiments, the tip of the catheter is coated in a radio-opaque substance in order to be visualized on fluoroscope imaging.

In exemplary embodiments, the balloon of the catheter is coated in a radio-opaque substance in order to be visualized on fluoroscope imaging.

In exemplary embodiments, the catheter is passed through a catheter sheath.

In exemplary embodiments, the catheter is first inserted into the duct, vessel, or space, followed by the catheter being advanced through the sheath in order to enter that same space.

In exemplary embodiments, the catheter contains a balloon that can be inflated at its tip, thereby blocking flow of solution through the entry site into the duct, vessel, or space.

In exemplary embodiments, the catheter contains radio-opaque material to confirm localization. In other words, the radio-opaque material may be used to track the location of the catheter during an injection process.

In exemplary embodiments, an adaptor can be positioned around the guidewires and pressure catheters inserted into a given lumen to further seal that lumen and prevent a fluid leak.

In exemplary embodiments, a multi-lumen catheter system with a forward-facing contrast injection lumen is preferentially utilized for nucleic acid or protein injection, in order to avoid biliary wall stress from lateral injection.

In exemplary embodiments, a catheter with a balloon proximal injection port is used for hydrodynamic injection of fluid in the proximal catheter direction.

In exemplary embodiments, a guidewire having a maximum of 0.018, 0.025, or 0.035 inches in diameter can be inserted into the second or third lumen.

In an aspect, the disclosure provides a method of sealing pressure in an unused lumen during injection of the gene delivery catheter system of claim 1, such that a cap or closed syringe is placed on the connector of the unused lumen during the injection in order to create a pressure seal and prevent fluid from escaping.

In exemplary embodiments, the catheter is used for introduction of nucleic acid or protein solutions into one of the following vessels or ducts: bile duct, gallbladder, pancreatic duct, urethra, urinary bladder, ureter, renal pelvis, lung airways, or vascular system.

In exemplary embodiments, the catheter is used to administer of nucleic acids or proteins at high fluid pressures in order to treat genetic, neoplastic, autoimmune, ischemic, metabolic, or inflammatory changes in the human body.

In an aspect, the disclosure provides a method of using the gene delivery catheter system of claim 1-32 is used for the treatment of endoscopic procedures of the gastrointestinal system, pulmonary system, or uretero-bladder system, as accessible intra-and/or extraluminal genetic, neoplastic or inflammatory changes in the human body, affording specific targeting of the intended organ.

In exemplary embodiments, the method is the treatment of liver, bile duct, pancreas, kidney, lung, heart or muscle diseases.

In exemplary embodiments, the catheter consists of two contrast injection lumens, one of which has a lateral opening proximal to the balloon, and the other of which injects forward-facing at the distal end of the catheter.

In exemplary embodiments, the catheter consists of 3 or 4 lumens, of which there is a single lumen only to detect pressure which terminates on the proximal aspect of the balloon, such that a substantial increase in pressure in fluid proximal to the balloon is indicative of a leak around the balloon during upstream injection.

In exemplary embodiments, the catheter consists of three lumens, wherein one of the lumens has a dual port to allow insertion of a guidewire and injection of fluid through a single large lumen at one time.

In exemplary embodiments, the catheter length (60 cm to at least 225 cm) is designed to accommodate both adult and pediatric endoscopic retrograde cholangiopancreatography (ERCP) procedures

In exemplary embodiments, the pressure sensor inserted into the multi-lumen catheter system is connected a pressure transducer, such that real-time pressure can be monitored during the injection

In exemplary embodiments, the catheter has 5 lumens, with the additional 5th opening proximal to the balloon to permit injection of contrast and priming of the biliary tree.

In exemplary embodiments, the catheter further includes a fifth lumen, wherein the fifth lumen includes an additional opening proximal to the balloon to permit monitoring of pressure via a transducer during injection to detect leakage around the balloon during injection upstream of the balloon.

In exemplary embodiments, the catheter consists of 3 or 4 lumens, wherein one of the 3 or 4 lumens is a single lumen configured only to detect pressure which terminates on the proximal aspect of the balloon, such that a substantial increase in pressure in fluid proximal to the balloon is indicative of a leak around the balloon during injection upstream of the balloon.

In exemplary embodiments, the distal tip has enhanced flexibility, due to reduction in caliber of the catheter in the distal quarter or third, or use of different materials., enabling maneuverability into the ductal system over a guidewire.

In exemplary embodiments, the balloon is spherical, cylindrical, or pear shaped to allow for effective and safe occlusion of the bile duct

In exemplary embodiments, the balloon is made of durable materials that permit occlusion of the duct wall yet effectively minimize friction within the ductal wall when intentionally maneuvering the balloon (e.g., silicone and the like). This material will permit the balloon to conform (tubularize) along the length of the bile duct wall as opposed to rupturing it.

In exemplary embodiments, the balloon is inflated to a size based on the caliber of the bile duct measured on imaging modality performed prior to the procedure.

In exemplary embodiments, the balloon is inflated to a size based on the caliber of the bile duct measured fluoroscopically during the procedure.

In exemplary embodiments, the balloon inflation is not inflated to a target size but is regulated by pressure within the balloon that ensures an adequate seal without being excessive.

In one aspect, the disclosure provides a gene delivery catheter system for performing endoscopic-mediated gene and protein delivery into liver, pancreas, and kidney organs, comprising a multi-lumen catheter having a catheter body having a substantially cylindrical shape and a substantially uniform in diameter along its longitudinal axis, and having three or four mutually independent lumens extending through the catheter body, wherein the multi-lumen catheter for insertion into a working channel of an endoscope is formed and dimensioned, characterized in that—the multi-lumen catheter contains:
(a) a lumen to partially deploy a fully covered self-expandable metallic stent,
(b) a second lumen for receiving a guidewire,
(c) a third lumen for receiving and transmitting a solution, and
(d) a fourth lumen for receiving a pressure catheter/transducer.

In exemplary embodiments, the second lumen and third lumen can be combined into a single lumen accomplishing both tasks, with the guidewire removed prior to nucleic acid or protein solution injection.

In exemplary embodiments, the catheter will only include a single lumen, which houses the stent to be partially delivered and permits nucleic acid or protein solution injection.

In exemplary embodiments, the partially deployed covered metallic stent remains attached to the catheter, allowing for ease of resheathing and removal from the bile duct.

In exemplary embodiments, the partially deployed covered metallic stent is made of nitinol and covered with silicone or polytetrafluorethylene (PTFE) membranes.

In exemplary embodiments, the partially deployed covered metallic stent when deployed will have a larger diameter than the duct to ensure tight wall apposition and adequate seal during injection of contrast or nucleic acid or protein solution injection.

In exemplary embodiments, the partially deployed covered metallic stent may have an olive tip at its distal tip of the catheter to allow atraumatic passage of the catheter into the targeted organ or tissue.

In exemplary embodiments, the partially deployed covered metallic stent may have an olive tip at its distal tip of the catheter that has an orifice that permits the passage of a guidewire.

In exemplary embodiments, the partially deployed covered metallic stent may have an olive tip at a distal tip of the catheter that has an orifice that permits passage of the injection of contrast agent or plasmid solution after the guidewire is removed.

In exemplary embodiments, the partially deployed covered metallic stent may have an olive tip at a distal tip of the catheter which can be retracted after partial stent deployment to the very tip of the catheter such that it resides in the stent to block the distal end of the catheter during the injection of contrast agent or plasmid solution to prevent backflow.

In exemplary embodiments, the lumen receiving the guidewire, the lumen transmitting and receiving fluid, and the lumen receiving the pressure sensor are all housed in the olive tip allowing for the guidewire to remain and pressure to be measured during injection of nucleic acid or protein solution.

In exemplary embodiments, the lumen receiving the guidewire and the lumen transmitting and receiving fluid at the olive tip are the same, with the guidewire being removed prior to injection of nucleic acid or protein solution.

In exemplary embodiments, the catheter is ingeniously designed to accommodate both adult and pediatric endoscopic retrograde cholangiopancreatography (ERCP) scopes, covering a range from 60 to at least 225 cm. This versatility ensures its effectiveness across diverse patient populations.

In exemplary embodiments, the catheter incorporates an adjustable balloon capable of inflation to various and diameters (6 to 25 mm, or greater) and lengths (10, 12, 15, 20, 25, 30, 35, 40, 45, 50 mm or greater mm). Balloon shapes post-inflation, include the conventional spherical balloon. To decrease the risk of bile duct wall injury and injury to surrounding tissue (hepatocytes, vasculature, etc.), longer (length along the catheter greater than the diameter of the inflated balloon—i.e., a cylindrical shape) length balloons can be used to increase the surface area the balloon contacts the bile duct wall, effectively spreading the forces of the occlusion balloon across a greater surface area, decreasing the risk of injury. Furthermore, longer balloons also allow for superior occlusion due to the lower risk of leakage around the balloon as a result of a larger surface area of occlusion. A longer balloon will also make easier the ability to occlude the cystic duct. Additionally, the longer balloon will be easier to see fluoroscopically. The balloon will be constructed from high-quality, durable materials that effectively minimize friction with the ductal wall. In one embodiment, the material will include silicone, latex, or nylon. The primary objective is to significantly reduce resistance encountered while manipulating the balloon into the ducts. Furthermore, this design seeks to enhance the overall longevity of the balloon throughout the procedures and to mitigate the risks of friction-induced damage or premature bursting during the procedures.

In exemplary embodiments, the balloon size selected to occlude the duct is estimated based on the cholangiogram, as well as prior imaging such as CT, MRI, US. One strategy is to inflate the balloon to 1.5×, 2×, 3×. 4× the diameter of the bile duct at the location of the balloon to ensure adequate occlusion. In one embodiment, the balloon will be inflated using an automated device that controls the pressure within the balloon. The pressure is regulated so that it is high enough to abut the bile duct wall and provide adequate seal, but no excessive to cause clinically significant injury to the bile duct. A feedback loop is established between sensors in the balloon and the inflation device to maintain the pressure within a specified range. In one embodiment, the pressure is kept between 60-70 mbar, with a tolerance of +/−10 mbar (equivalent to a range of 0.87-1.02 psi). Other embodiments have pressures that maybe range lower or higher. To reposition the balloon within the ductal system, the pressure will be decreased by 15% of the set value. This reduction in pressure allows the balloon to be inflated sufficiently for easy manipulation without exerting excessive pressure on the ductal walls, thereby preventing injury to the ductal system.

In exemplary embodiments, the balloon's size selection depends on the species; empirical data demonstrates that smaller diameter balloons are safer for porcine bile ducts, while larger diameter balloons are more effective in primate subjects. This is related to the more flexible bile ductal system in primates compared to the pigs.

In exemplary embodiments, a pear-shaped balloon may also provide benefit, particularly in situations where the downstream bile duct (distal common hepatic duct and proximal common bile duct) is greater in diameter than the more upstream common hepatic duct or intra hepatic ducts. Furthermore, as the bile duct wall dilates as a result of hydrodynamic injection more prominently around the tip of the catheter, the pear shape will help maintain the seal. In one embodiment, the balloon is radiolucent with radiopaque markers placed on it to enhance visibility during imaging.

In exemplary embodiments, inflation of the balloon in the common hepatic duct (above the cystic duct opening that connect the gall bladder to the bile ducts) ensures homogenous delivery of DNA solutions to the entire liver (FIGS. 19A-19C). In one embodiment, selective inflation of the balloon in the right or left intrahepatic ductal systems provides support from the surrounding liver tissue and prevents duct injury, though the balloon's shape will distort and tubularize if inflated significantly beyond the caliber of the duct (FIGS. 19D-19E).

In exemplary embodiments, the catheter will be made of only two lumens (FIGS. 22A-22B), the goal is to have one large lumen to adapt a fully covered self-expandable stent (SES), and a smaller lumen to adapt the pressure sensor. The purpose of this catheter design is to allow the delivery of a fully covered SES to oppose the wall of the ductal system (e.g., the bile duct) thus occluding the opening of the cystic duct and preventing leakage into the gall bladder when used for biliary injection. The SES will also maintain an even distribution of pressure on the ductal walls to avoid excess pressure on one section of the ductal system. This even distribution of pressure will prevent marked flow turbulence during injection that would result from the change in the diameter of the ductal system in case of uneven distribution of pressure. The smaller second lumen of the catheter will contain a pressure sensor to measure the pressure inside the ductal system during the injection of the DNA solution.

In exemplary embodiments, when pressure measurement is not required, the catheter will have only one large lumen and the second lumen will be removed. This catheter design will also simplify the manufacturing process of the catheter with only two or in some embodiments one lumen.

In exemplary embodiments, the SES can be made from nitinol wire alloy with a shape memory that will adapt to the ductal system at body temperature.

In exemplary embodiments, the SES will be covered with silicone or polytetrafluorethylene (PTFE) membranes. Using these membranes is effective in preventing leaks of contrast agents or plasmid solutions. These membranes are also commonly used to manufacture fully covered stents which will simplify the manufacturing process. The SES will have a larger diameter than the ductal system to allow tight opposition of the wall and prevent leakage of the contrast agent or the DNA solution.

In exemplary embodiments, the SES diameter will be 10-20% larger than the average diameter of the ductal system. The SES will be carefully folded around a guiding sheath inside the larger catheter lumen. The guiding sheath will have an internal lumen that will be used as an injection port for both the contrast agent and the plasmid solution and to pass a guidewire (FIGS. 20A-20B).

In exemplary embodiments, the guiding sheath will have an olive tip at its distal end (near the liver) (FIG. 21A). The olive tip end of the sheath will allow atraumatic passage of the catheter into the targeted organ or tissue. Also, the olive tip may block the distal end of the catheter during the injection of contrast agent or plasmid solution to prevent backflow (FIGS. 21A-21C).

In exemplary embodiments, during an endoscopy or interventional radiology procedure, the catheter is advanced, and contrast may be serially injected to elucidate the branching network. The guidewire may direct the catheter into the desired location at the ductal system (e.g., the common bile duct or the common hepatic duct). Once the appropriate catheter position is reached at the common bile duct, the SES may be partially deployed allowing the stent to expand and oppose the ductal wall. Once the distal half of the stent is deployed, it will have a funnel shaped configuration. This configuration will prevent backflow of contrast or the plasmid solution during injection. In one embodiment, when the guiding sheath has an olive tip at the distal end, the sheath will be pulled back through the opened segment of the SES until the olive tip reaches the distal end of the catheter to block it and prevent backflow of contrast or plasmid solution. The guidewire will be removed, and a contrast agent will be injected to ensure that the cystic duct opening is totally occluded by the SES and there is no leakage inside the gall bladder. In one embodiment, the guidewire will be left in place while injecting the solution through a separate port (FIGS. 21A-21C).

In exemplary embodiments, no contrast will be injected and only the guidewire will be used to guide the position of the catheter. The DNA solution will then be injected at X ml/second, and the pressure inside the lumen of the bile duct will be measured using the pressure sensor, when required. After the DNA solution injection, the SES will be recaptured back into the catheter. In one embodiment, when the guiding sheath has an olive tip at the distal end, the sheath will be pulled back until the olive tip reaches the distal end of the catheter. The catheter will be removed from the common bile duct and retrieved back into the endoscope.

In an aspect, the disclosure provides a gene delivery catheter, which consists of three lumens, wherein the two of the lumens are forward-facing with one slightly larger than the other, allowing for insertion of pressure catheter and/or guidewire in one lumen, and injection of fluid in the other.

### Definitions

To facilitate an understanding of the present disclosure, a number of terms and phrases are defined below:

Unless specifically stated or obvious from context, as used herein, the term “about” is understood as being within a range of normal tolerance in the art, for example within 2 standard deviations of the mean. About can be understood as within 50%, 45%, 40%, 35%, 30%, 25%, 20%, 15%, 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

By “agent” is meant any small molecule chemical compound, antibody, nucleic acid molecule, polypeptide, or fragments thereof.

By “ameliorate” is meant decrease, suppress, attenuate, diminish, arrest, or stabilize the development or progression of a disease (e.g., a Wilson's disease phenotype).

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “control” is meant a standard or reference condition.

By “disease” is meant any condition or disorder that damages or interferes with the normal function of a cell, tissue, or organ.

By “fragment” is meant a portion of a polypeptide or nucleic acid molecule. This portion contains, preferably, at least 5%, 10%, 20%, 30%, 40%, 50%, 60%, 70%, 80%, or 90% of the entire length of the reference nucleic acid molecule or polypeptide. A fragment may contain 5, 10, 20, 30, 40, 50, 60, 70, 80, 90, or 100, 200, 300, 400, 500, 600, 700, 800, 900, 1000, 2000, 3000, 4000, 5000 or more nucleotides or amino acids.

A “gene therapy agent” is to be understood as meaning a DNA composition (e.g., a recombinant nucleic acid including a gene and/or associated elements, e.g., an enhancer, a promoter, etc., for its transcription and/or translation in vivo) for generating prophylaxis and/or treatment of a genetic disorder.

By “isolated polynucleotide” is meant a nucleic acid molecule (e.g., a DNA, an mRNA, a cDNA, and the like) that is free of the genes from which, in the naturally occurring genome of the organism, the nucleic acid molecule of the disclosure is normally associate or derived. The term therefore includes, for example, a recombinant DNA (e.g., including a genomic DNA or cDNA coding for a ATP7B gene, as well as associated regulatory components such as, for example, an enhancer(s), a promoter, 5′ and/or 3′ untranslated regions (UTRs), and the like) that may be incorporated into: a vector, or an autonomously replicating plasmid or virus, or into the genomic DNA of a prokaryote or eukaryote, or into a polynucleotide that exists as a separate molecule (for example, a cDNA or a genomic or cDNA fragment produced by PCR or restriction endonuclease digestion, or a naked DNA construct such as a plasmid or cosmid or linear DNA) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the disclosure that has been separated from components that naturally accompany it. Typically, the polypeptide is isolated when it is at least 60%, by weight, free from the proteins and naturally occurring organic molecules with which it is naturally associated. Preferably, the preparation is at least 75%, more preferably at least 90%, and most preferably at least 99%, by weight, a polypeptide of the disclosure. An isolated polypeptide of the disclosure may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

Unless specifically stated or obvious from context, as used herein, the term “or” is understood to be inclusive. Unless specifically stated or obvious from context, as used herein, the terms “a,” “an,” and “the” are understood to be singular or plural.

The term “patient” or “subject” refers to an animal which is the object of treatment, observation, or experiment. By way of example only, a subject includes, but is not limited to, a mammal, including, but not limited to, a human or a non-human mammal, such as a non-human primate

As used herein, the terms “prevent,” “preventing,” “prevention,” “prophylactic treatment,” and the like, refer to reducing the probability of developing a disease or condition (e.g., Wilson's disease) in a subject, who does not have, but is at risk of or susceptible to developing the disease or condition (e.g., Wilson's disease).

“Primer set” means a set of oligonucleotides that may be used, for example, for PCR. A primer set would consist of at least 2, 4, 6, 8, 10, 12, 14, 16, 18, 20, 30, 40, 50, 60, 80, 100, 200, 250, 300, 400, 500, 600, or more primers.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

By “reduces” is meant a negative alteration of at least 10%, 25%, 50%, 75%, or 100%.

By “reference” is meant a standard or control condition.

A “reference sequence” is a defined sequence used as a basis for sequence comparison (e.g., a wildtype ATP7B gene sequence). A reference sequence may be a subset of, or the entirety of, a specified sequence; for example, a segment of a full-length cDNA or genomic sequence, or the complete cDNA or genomic sequence. For polypeptides, the length of the reference polypeptide sequence will generally be at least about 10-5,000 amino acids, 10-4,000 amino acids, 10-3,000 amino acids, 10-2,000 amino acids, 10-1,500 amino acids, 10-1,000 amino acids, 10-500 amino acids, or 10-100 amino acids. Preferably, the length of the reference polypeptide sequence may be at least about 10-50 amino acids, more preferably at least about 10-40 amino acids, and even more preferably about 10-30 amino acids, about 10-20 amino acids, about 15-25 amino acids, or about 20 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or there between.

Nucleic acid molecules useful in the methods of the disclosure include any nucleic acid molecule that encodes a polypeptide of the disclosure, or a fragment thereof. Such nucleic acid molecules need not be 100% identical with an endogenous nucleic acid sequence, but will typically exhibit substantial identity (e.g., 100%, 99%, 98%, 97%, 96%, 95%, 94%, 93%, 92%, 91%, or 90%). Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences, or portions thereof, under various conditions of stringency. (see, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30 °C, more preferably of at least about 37 °C, and most preferably of at least about 42 °C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30 °C in 750 mM NaCl, 75 mM trisodium citrate, and 1% SDS. In a more preferred embodiment, hybridization will occur at 37 °C in 500 mM NaCl, 50 mM trisodium citrate, 1% SDS, 35% formamide, and 100 μg/ml denatured salmon sperm DNA (ssDNA). In a most preferred embodiment, hybridization will occur at 42 °C in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25 °C, more preferably of at least about 42 °C, and even more preferably of at least about 68 °C. In a preferred embodiment, wash steps will occur at 25 °C in 30 mM NaCl, 3 mM trisodium citrate, and 0.1% SDS. In a more preferred embodiment, wash steps will occur at 42 °C in 15 mM NaCl, 1.5 mM trisodium citrate, and 0.1% SDS. In a more preferred embodiment, wash steps will occur at 68 °C in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid or nucleotide sequence (for example, any one of the amino acid or nucleotide sequences described herein). Preferably, such a sequence is at least 60%, at least 70%, at least 80%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, or at least 100% identical at the amino acid sequence or nucleic acid sequence used for comparison (e.g., wildtype ATP7B).

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

As used herein, the terms “treat,” “treated,” “treating,” “treatment,” and the like refer to reducing or ameliorating a disorder and/or symptoms associated therewith (e.g., Wilson's disease). It will be appreciated that, although not precluded, treating a disorder or condition does not require that the disorder, condition, or symptoms associated therewith be completely eliminated.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

The above-mentioned and other features and advantages of the present disclosure will be better understood when reading the following detailed description taken together with the following drawings in which:

FIG. 1 shows an illustration of a hydrodynamic injection catheter having a distal end or tip with a blunt leading edge including an injection orifice in fluid communication with an injection lumen. The blunt leading edge is generally oriented at 90 ° relative to the longitudinal axis of the injection catheter.

FIG. 2A shows an example of cross-section view of the catheter showing the four different lumens is presented. In this example, the injection lumen occupies over 50% of the total diameter of the catheter. The other three lumens occupy smaller diameters to their minimal diameter for the pressure catheters and guidewires sold on the market. The balloon catheter lumen only transmits air alone and optimally fits in a small residual diameter in the catheter. In preferred embodiments, the area of the catheter in gray is solid or sealed off, so no fluid enters this space by retrograde flow.

FIG. 2B shows an example of cross-section view of the catheter showing the four different lumens is presented. In this example, the injection lumen is equal size to the guidewire lumen. This allows for either lumen to be utilized for nucleic acid injection, if the guidewire is removed prior to nucleic acid injection through the lumen.

FIG. 2C shows an example of cross-section view of the catheter showing the four different lumens is presented. In this example, the largest lumen contains the guidewire, which is subsequently removed so that nucleic acid solution can be injected through that lumen. Another smaller channel is utilized for contrast injection, such that the two lumens do not cross-contaminate each other.

FIG. 2D shows an example of a quadruple lumen catheter cross-section is depicted. Here, the injection lumen is a semi-circle. The guidewire lumen is the circle. Two smaller circles are for the air balloon and either pressure catheter or contrast injection.

FIG. 2E shows an example of a quadruple lumen catheter cross-section area is depicted, wherein circular lumens of designated size for a guidewire and a commercial pressure sensor are provided. The remaining cross-section area is designed in shapes that utilize all available cross-section area, while leaving consistent reinforcement apart from the other lumens. The air balloon lumen is a smaller triangle, while the other cross-section is for DNA solution injection and contrast injection. The total diameter of the designated catheter is 8 French.

FIG. 2F shows an example of a quadruple lumen catheter cross-section is depicted. The three lumens for air balloon, guidewire, and pressure sensor are the same size and shape as the example cross-section depicted in FIG. 2E. The main difference here is that the injection lumen is now oval-shaped, while possessing almost the same cross-sectional area. The oval shape should help maintain laminar flow through the system. In other embodiments of this cross-section, the injection lumen is circular in shape in order to maintain perfect laminar flow and maintain ease of manufacturing.

FIG. 2G shows the cross-sectional area for the dual contrast injection port for the balloon catheter is depicted. This catheter leverages the ability to inject contrast above and below and inflated balloon simultaneously, such that exact localization within the vessel or ductal system could be obtained at any given time. The handle for this catheter is depicted in FIG. 8. In this cross-section, a large lumen is shared by the DNA/contrast/guidewire, which has the benefit of affording maximal size for tolerating hydrodynamic pressure. A separate contrast injection lumen for antegrade or backwards contrast injection is also shown. This lumen has significantly smaller diameter, since it does not need to tolerate large injection pressure and can be injected slowly. A pressure sensor lumen of the minimal size is already provided, along with the air balloon lumen of minimal size.

FIGS. 3A-3C depict exemplary embodiments of the catheter. FIG. 3A shows where the fluid will exit in the forward direction through the distal end of the catheter past the inflated balloon. FIG. 3B shows the injection port exits on a lateral aspect of the catheter proximal to the balloon, such that the fluid direction flow proceeds toward the proximal end of the catheter. FIG. 3C shows an exemplary lateral injection lumen is depicted, wherein the lumen injects to the side of the catheter before reaching the distal end of the catheter.

FIG. 4 illustrates dimensions of the balloon at the end of the catheter, with examples of the diameters of the balloon inflation being provided. The catheter tip would extend past the tip of the balloon. Examples of the distances of the tip of the catheter, where solution exits, range from 1-3 cm, and is minimally 0.5 cm in distance.

FIG. 5 shows a diagram of a spherical balloon that could be employed in the catheter is depicted. The blunt tip of the catheter is depicted as well. The balloon here is made of polyurethane for flexibility and strength.

FIG. 6 shows a diagram of an exemplar quadruple lumen catheter is depicted, wherein the handle for the four lumens is depicted, along with an illustration of the balloon and tip of the balloon.

FIG. 7 shows examples of the proximal ends of the of the quadruple catheter. These ends would be closest to the user and operated for clinical use.

FIG. 8 illustrates a catheter handle for a quintuple port, quadruple lumen catheter for hydrodynamic injection is depicted. This catheter leverages the ability to inject contrast above and below and inflated balloon simultaneously, such that exact localization within the vessel or ductal system could be obtained at any given time. This is accomplished through a proximal contrast channel (3) opening up below the balloon with a side facing outlet channel. The DNA/Contrast injection lumen and guidewire lumen are shared in this model to save space, such that contrast and the guidewire would be in place at the same time. It is envisioned that a dual port would connect to a single lumen to allow access for this purpose. Besides lumen 3 and 5, all other lumens/ports would be forward facing in this system.

FIG. 9 shows a quadruple port, triple lumen catheter for hydrodynamic injection is depicted. The rationale for this catheter is that the total diameter catheter size could be made smaller if the number of lumens was reduced from 4 to 3. This is especially advantages for renal applications, where the catheter diameter must be small to fit into the cystoscope working channel. This reduction in lumen number and catheter size is accomplished through sharing the guidewire and contrast injection through a single large lumen at one point. This lumen had a sufficiently large diameter to accommodate both substances at one time. For DNA injection itself, the guidewire would be pulled out, and injection allowed to proceed with the additional port closed or sealed.

FIG. 10 shows dimensions of the diameter of the catheter, as well as examples where the radio-opaque markings can be located, just before the tapering of the catheter. The dimensions of the catheter are such that it could easily fit into the bile ducts, pancreatic ducts, and ureter systems of the human body, among other potential vessels.

FIG. 11 shows an experimental setup for testing the pressure sensor inside a catheter is depicted.

FIG. 12 shows a simple dual lumen catheter was constructed to allow for testing of the pressure catheter inside of it, to assess the location the pressure sensor tip needs to be. The image on the left shows the catheter without the pressure sensor in it, while on the right we see the pressure sensor in place.

FIG. 13 is a graph depicting the location of pressure sensor tip was tested in multiple locations through the designated pressure sensor lumen in the catheter. It was observed that the pressure sensor could be pulled 30 cm inside the catheter without any significant change in the pressure sensor readings observed.

FIG. 14 shows an exemplary catheter having a steerable end that can be moved with a wire, allowing it to be guided through different ductal systems in the body. An example of how the catheter tip could be moved is depicted. In this example, the wire that pulls the distal end of the catheter runs underneath the balloon

FIG. 15 shows an example of how the different elements can exit the distal end of a tapered catheter are depicted. In this embodiment, the guidewire exits out of the catheter to help with localization in the vessel of interest. Meanwhile the pressure catheter is also advanced beyond the catheter in order to evaluate the pressure in real time. The DNA solution exits in a forward direction, rushing alongside the guidewire and pressure catheter. The lumens here are depicted in gray, with the relative size of the injection lumen being the largest.

FIG. 16 shows an example of a catheter design with a tapered distal tip and side wall injection is presented. In this example, all four lumens a travel through the body of the lumen, and then at the very distal tip, there injection lumen ends early with an exit on the lateral aspect of the catheter. In other embodiments, the injection lumen will exit in a forward direction. Of note, at the very distal tip, it is past where the balloon has been inflated, such that only the other three lumens remain. Radio-opaque markings are included at the end of the catheter.

FIG. 17 shows exemplary examples of different tapering options of the catheter are depicted. The tapering scheme can be acute, or more gradually sloping. In other embodiments, the catheter can have a blunted catheter end wherein no tapering occurs.

FIG. 18 shows the proximal handle of the catheter will have openings for four different lumens, with connectors attached to all lumens for inserting of syringes and tubing, along with the ability of each lumen to be seal. An example of one version of how the different catheters could be oriented is provided relative to each other.

FIGS. 19A-19E show the anatomy of the biliary ductal system is shown (FIG. 19A). A deflated balloon catheter is shown passing through the common bile duct into the common hepatic duct, with contrast (dark grey) visible around the catheter (FIG. 19B). A balloon catheter is shown passing through the common bile duct into the common hepatic duct with an inflated balloon (FIG. 19C). Adequate sealing ensures injection only towards the liver above the balloon, while contrast remains below in the bile ductal system. A balloon catheter is shown passing through the common bile duct into the left hepatic duct with an inflated balloon (FIG. 19D). Adequate sealing ensures injection only towards the left liver lobe above the balloon, while contrast remains below in the bile ductal system. A balloon catheter is shown passing through the common bile duct into the right hepatic duct with an inflated balloon (FIG. 19E). Adequate sealing ensures injection only towards the right liver lobe above the balloon, while contrast remains below in the bile ductal system.

FIGS. 20A-20B illustrate an exemplary self-expandable stent partially deployed with a catheter passing through it (dark blue color) and a guidewire (thin yellow line) passing inside the catheter (FIG. 20A). The withdrawal of the catheter along with a guidewire and the injection of a solution (cyan, e.g., contrast or DNA solution) is shown (FIG. 20B).

FIGS. 21A-21C illustrate an exemplary self-expandable stent is shown partially deployed with a catheter passing through it (blue color) and a guidewire passing inside the catheter (FIG. 21A). The catheter features an olive tip distal end in this embodiment. The withdrawal of the catheter positions the olive tip end, to prevent the backflow of contrast or DNA solution during injection (FIG. 21B). The injection of a solution (contrast or DNA solution) while the olive tip end is positioned prevents backflow beyond the stent opening (FIG. 21C).

FIGS. 22A-22B illustrate an exemplary transverse section (FIG. 22A) and a side view (FIG. 22B) of the catheter are shown, including two lumens: a large one for the self-expandable stent and a small one for the pressure sensor.

FIGS. 23A-23B is a photograph showing the handle of the catheter with a Y Luer connector to permit both guidewire passage and injection of a solution through a single lumen (FIG. 23A). Photograph showing the handle of the catheter without the Y Luer connector (FIG. 23B).

FIGS. 24A-24D show a series of graphs depicting pressure changes from a porcine trial. The graphs display injection volume and flow rate (2 ml/s) with the pressure sensor placed at the catheter tip (FIG. 24A), and 5 cm (FIGS. 24B), 10 cm (FIG. 24C), and 100 cm (FIG. 24D) from the catheter tip towards the handle. Injection volumes were 30 mL, 30 mL, 28 mL and 34 mL, respectively.

### DETAILED DESCRIPTION

The present disclosure relates to an apparatus and system for hydrodynamic injection of gene therapy agents. More particularly, the present disclosure relates to an apparatus and system for hydrodynamic injection of gene therapy agents into large animals and humans. As described in detail below, the present disclosure is based, at least in part, on the surprising discovery that a hydrodynamic catheter system having specific lumen configurations may be used to deliver gene therapy agents (e.g., one or more recombinant nucleic acids and/or one or more proteins) to a patient. An exemplary hydrodynamic catheter system may include three or more lumens such as, for example, a pressure catheter lumen (e.g., to monitor injection of a gene therapy agent), a guidewire lumen, an injection lumen to inject a solution (e.g., a gene therapy agent, a contrast agent, etc.), or a balloon catheter lumen (e.g., to inflate a balloon at the distal tip of a catheter).

According to the techniques herein, the injection lumen may be maximized in size or modified in circumferential shape relative to the other lumens in order to reduce wall stress and optimize flow rate within the catheter during hydrodynamic injection.

### Overview

As noted above, there are significant disadvantages to using viral-based (e.g., AAV, LV, etc.) gene therapy agents including: size constraints on the length of a nucleotide sequence that may be packaged within the viral-based vector, potential to integrate into the host genome, potential to generate a host immune response, complex to make and manufacture. The ability to efficiently introduce non-viral gene therapy vectors into a large animal or human subject would have a number of advantages such as, for example: non-viral gene therapy vectors exist as an episome without being integrated into the host genome; there is no host immune response or host side effects caused by the administration of non-viral gene therapy vectors, producing non-viral gene therapy vectors is quite cheap, etc. (see, e.g., Li L, Petrovsky N. Molecular mechanisms for enhanced DNA vaccine immunogenicity. Expert Rev Vaccines. 2016; 15(3):313-29).

Hydrodynamic injection has been an efficient method by which to introduce non-viral gene therapy vectors into mice. Hydrodynamic injection consists of using high pressure to create temporary pores in a cell membrane to allow DNA to cross the cell membrane and enter into cells/tissues/organs. While this technique has proven very successful in murine models, the scaling of hydrodynamic injection into larger animals (e.g., humans) has proven to be very challenging. The majority of methods of hydrodynamic injection in large animals revolve around isolating the vasculature of specific organs and injecting the fluid locally. In order to accomplish this, various different interventional radiology, flexible endoscopy, bronchoscopy, cystoscopy or ureteroscopy strategies could be employed.

Hydrodynamic injection relies heavily on the design of the catheter used to deliver gene therapy vectors to the tissues or organs of interest. Catheters are used during many different medical procedures today. For example, catheters may be placed inside blood vessels in order to remove clots for the management of acute coronary syndrome, heart attacks, or stroke. Another example leverages catheters inserted into the biliary system via endoscopic retrograde cholangiopancreatography (ERCP) to remove bile stones that may be lodged in the biliary system, which may result in jaundice and abdominal pain. Catheters may also be inserted into the ureter to remove kidney stones blocking urine outflow, which may cause pain and also risk infection to a patient. Depending on the intended clinical use, catheters may have special modifications or channels (e.g., lumens) to endow them with specific functions. For example, surgical biopsy tools may be inserted through small lumens in the catheters in order to acquire tissue and pull it back for sampling (Endosc Int Open. 2018 Aug; 6(8): E984-E988). As another example of clinical use, catheters may be used to deliver shock treatments (lithotripsy) in order to break up kidney stones that might be causing obstructions in a procedure termed intravascular lithotripsy. Unfortunately, current catheter designs have proven ineffective for efficient delivery of gene therapy vectors in large animals. Despite the many different types of catheters that exist today in medical practice, all extent catheters have significant limitations for use in delivering gene therapy vectors via hydrodynamic injection because they were not designed specifically for the hydrodynamic injection of nucleic acid solutions at high pressures. For example, the CleverCut3V Distal Wireguided (Olympus Medical) catheter, which is principally used for steering and placing a guidewire into the ampulla of Vater—the opening of the biliary orifice—does not have the ability to contain a DNA solution or experience fluid pressure.

The present disclosure used the Multi-3 V Plus, triple-lumen, single-use stone extraction balloon catheter (Olympus Medical) for hydrodynamic injection. The balloon on the Multi-3 V Plus catheter can be inflated to three different sizes (e.g., 8.5 mm, 11.5 mm, and 15 mm), and was able to withstand the hydrodynamic pressure in all experiments. The Multi-3 V Plus catheter only has three lumens (e.g., an air/balloon lumen, a guidewire lumen that is forward facing, and a contrast injection lumen that is side facing). Disadvantageously, the Multi-3 V Plus catheter contains no dedicated lumen for a pressure sensor, which could theoretically only fit inside the guidewire lumen, or for DNA injection. Additionally, the guidewire lumen of the Multi-3 V Plus catheter is the only sizable lumen that could be used for fluid injection, which presents a severe disadvantage because the guidewire must be removed prior to DNA injection. The contrast lumen is useful for localization of the catheter but cannot accommodate either the pressure catheter or the guidewire. The contrast lumen is also constrained in having a side-facing injection port, which places pressure onto the duct or vessel wall, thereby risking lumen wall rupture.

Accordingly, the Multi-3 V Plus catheter has significant disadvantages for use in a hydrodynamic injection system because it is not able to monitor pressure and inject DNA at the same time. These deficiencies makes sense because the catheter was not designed for hydrodynamic injection.

According to one or more embodiments of the disclosure, the techniques herein provide catheters and systems to facilitate hydrodynamic injection for gene therapy. An exemplary embodiment of a catheter disclosed herein may include a balloon to prevent injected fluid from leaking in at least one direction within a vessel space. For example, when the catheter's balloon is inflated, the injected fluid solution is directed to move in the desired direction toward the tissue/organ of interest. The techniques herein provide that an exemplary balloon is able to withstand the hydrodynamic pressure associated with a given injection protocol. Additionally, the shape of the balloon may be custom tailored (e.g., spherical, cylindrical, etc.) to the particular injection protocol to sufficiently seal the duct or vessel in the target tissue or organ of interest. Accordingly, the techniques herein provide balloon designs that have the correct dimensions and shapes to create an effective seal around the vessel or duct in the tissue or organ of interest that is being targeted in a particular hydrodynamic injection protocol.

An exemplary embodiment of the catheter design disclosed herein may include a variety of imaging modalities (e.g., C-arm mediated x-rays, fluoroscopy, and the like) to aid in localizing the catheter within a specific portion or space of a vessel, duct, lumen, etc., prior to injection. This may involve the use of radio-contrast agents which may be injected through the catheter. Additionally, the techniques herein provide that radio-opaque guidewires with small diameters may also be utilized to help localize the catheter in a particular vessel, duct, lumen and/or a given orifice of interest. An exemplary embodiment of the catheter design disclosed herein may include modalities for monitoring the pressure of hydrodynamic injection in real time.

Exemplary embodiments of the catheter design disclosed herein may include bespoke designs and configurations that are specifically tailored for hydrodynamic injection into particular vessels/ducts/lumens/orifices in particular target tissues and/or organs. In other words, the catheter embodiments disclosed herein may be optimally designed for the vessels/ducts/lumens/orifices that are being targeted for the hydrodynamic gene delivery. Exemplary embodiments disclosed herein provide catheter designs and configurations that may be optimally suited for hydrodynamic injection biliary system (e.g., the common hepatic duct, the common bile duct, the cystic duct, the right hepatic duct, and the left hepatic duct), the pancreatic duct (e.g., the duct of Wirsung and the accessory pancreatic duct), and the ureters. According to the techniques herein, the catheter may be configured to fit within the duct without causing undue trauma to the walls. Additionally, exemplary catheter designs disclosed herein may be modified so as to be used with commercially available endoscopes, duodenoscopes, cystoscopes, and bronchoscopes.

Unfortunately, there is no catheter currently available that provides all the different features required for a hydrodynamic injection procedure. The most complex catheters sold on the market today are generally double or triple lumen catheters and are custom-made for specific medical applications. Moreover, aside from a given number of lumens or ports, the individual lumens provided in prior art catheters are not suitable for hydrodynamic injection. For example, prior art catheter lumens are not configured to be able to handle high-pressure hydrodynamic injections. The guidewire lumen can often be the largest lumen in many cases, wherein a smaller lumen used for fluid injection. Thus, all catheters on the market are sub-optimal in addressing specific capabilities for hydrodynamic injection and there exists no previous device or technology that has been designed for this purpose.

As described in further detail below, the techniques herein provide embodiments of a hydrodynamic injection catheter including quadruple ports and/or lumens which has been specifically designed for use in the delivery of nucleic acid and/or protein solutions at high pressures through vessels/lumens/ducts/orifices in the body to deliver the nucleic acid and/or protein solutions into the tissues and/or organs of interest.

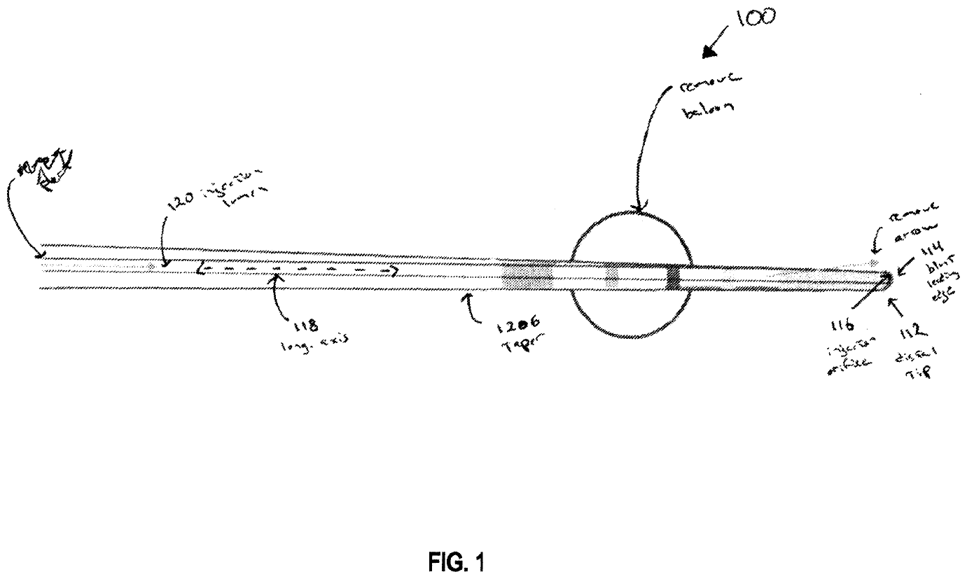

Referring to FIG. 1, a hydrodynamic injection catheter 100 may have a distal end or tip 112 with a blunt leading edge 114 including an injection orifice 116 in fluid communication with injection lumen 120. Blunt leading edge 114 is generally oriented add 90° relative to the longitudinal axis 118 of the hydrodynamic injection catheter 100. To facilitate the ability to insert hydrodynamic injection catheter 100 into a vessel/duct/lumen/orifice of a target tissue/organ, catheter 100 may include a taper 126, which generally may have a conical cross-section, that distally converges on distal tip 112.

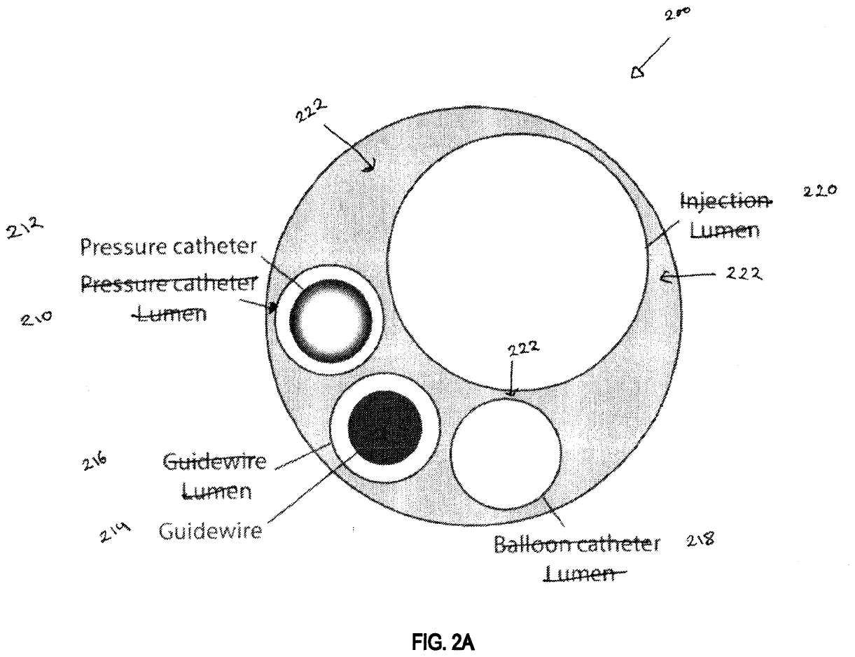

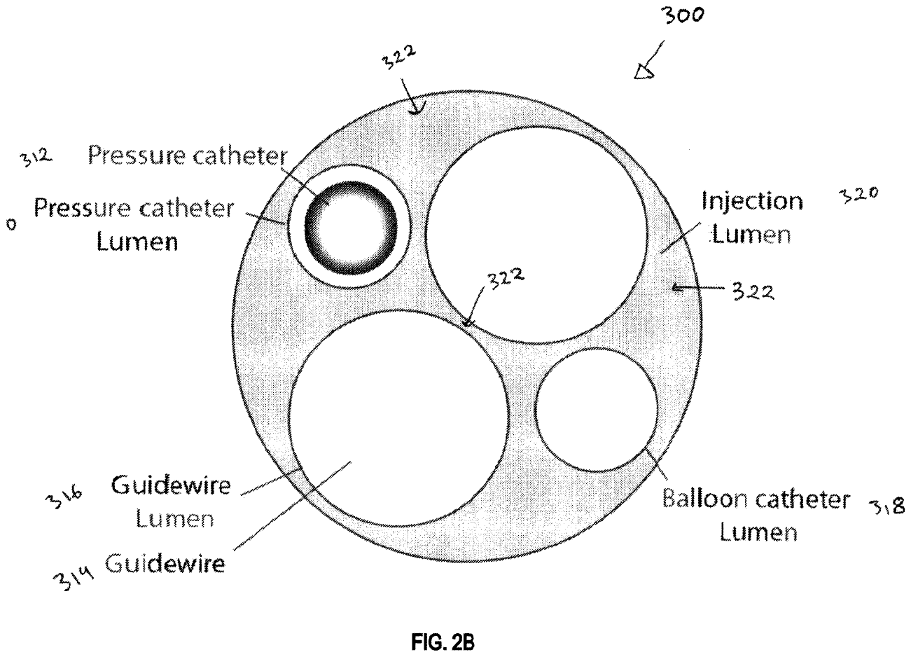

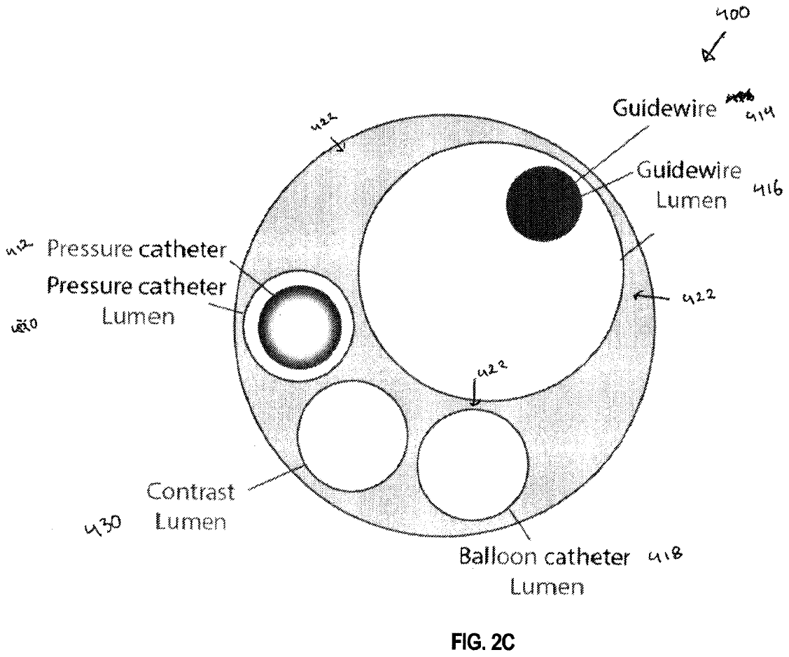

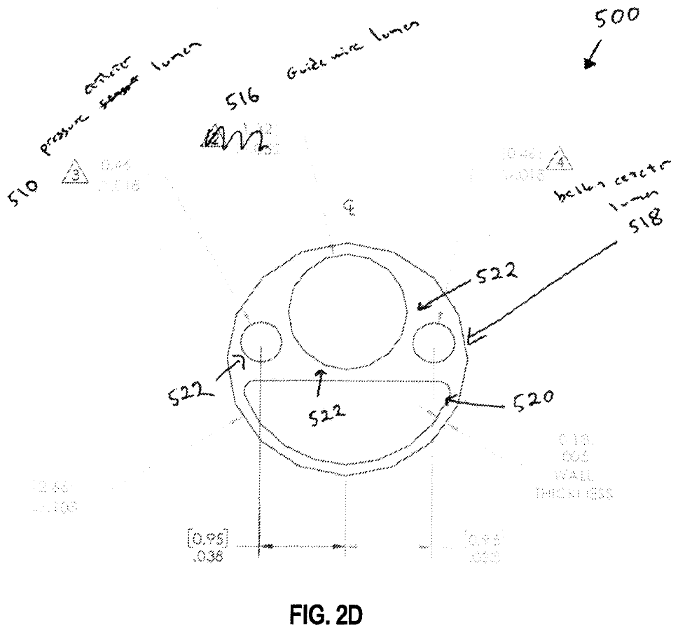

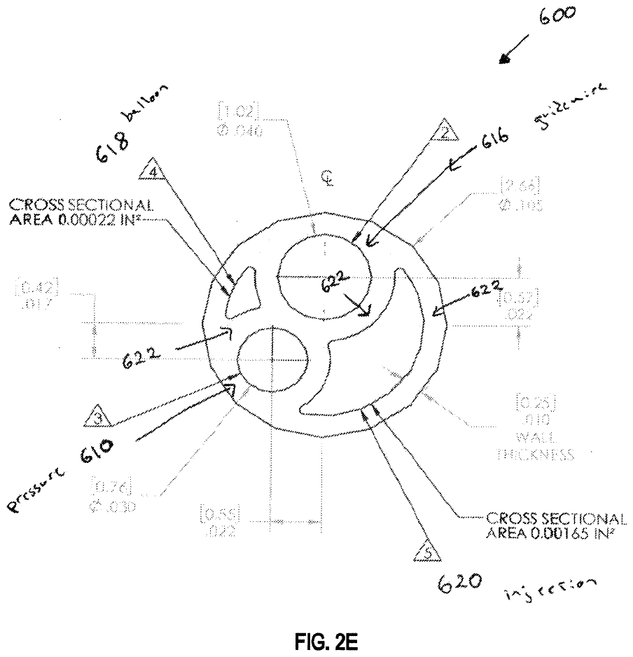

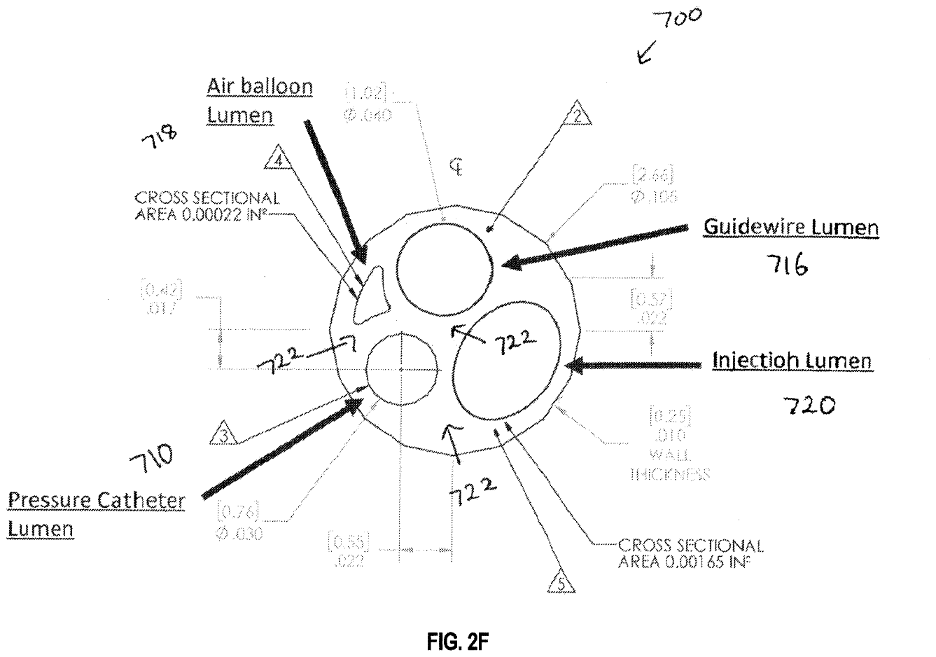

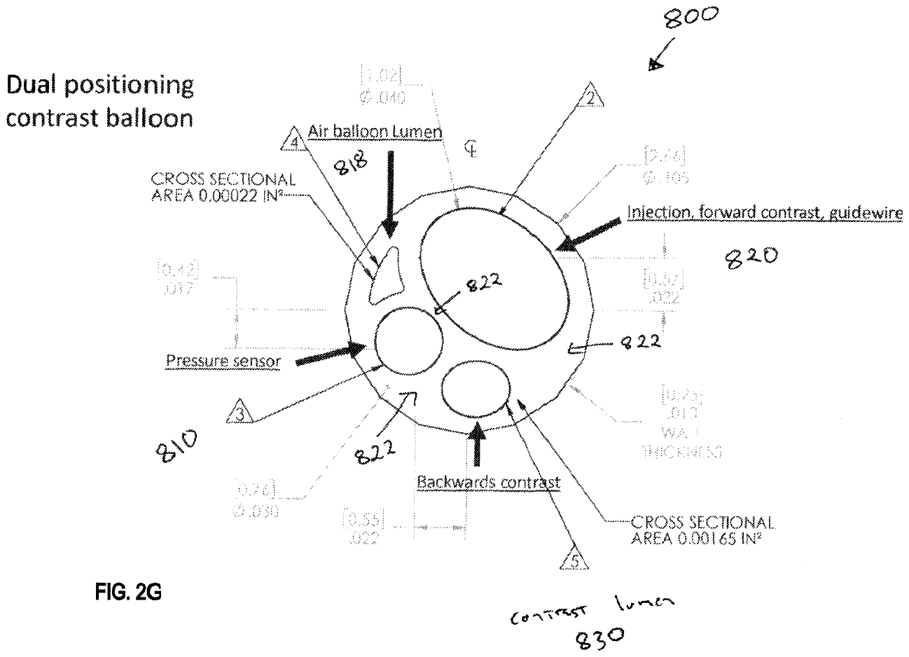

FIG. 2A is a cross-section of an embodiment of hydrodynamic injection catheter 100 having lumen configuration 200 which may include four different lumens such as, for example, a pressure catheter lumen 210, a guidewire lumen 214, a balloon catheter lumen 218, and an injection lumen 220. Pressure catheter lumen 210 may include pressure catheter 212. Similarly, guidewire lumen 216 may include guidewire 214, which is generally sizable compared to the pressure catheter. Lumen configuration 200 presents a cross-sectional profile in which injection lumen 220 is significantly larger than pressure catheter lumen 210, guidewire lumen 216, and balloon catheter lumen 218. For example, injection lumen 220 may occupy approximately 50% of the cross-sectional area of hydrodynamic injection catheter 100, while pressure catheter lumen 210, guidewire lumen 216, and balloon catheter lumen 218 may each occupy approximately 15% of the cross-sectional area of hydrodynamic injection catheter 100. FIG. 2A depicts the cross-sectional areas of pressure catheter lumen 210, guidewire lumen 216, and balloon catheter lumen 218 as approximately equal; however, the relative cross-sectional areas of each of these four lumens is representative and may vary between about 5% and about 10%. The relative sizes and positions of the lumens shown in FIG. 2A are representative and are not to be construed as being limiting with respect to the cross-sectional area or diameter or position of any of the four depicted lumens. For example, the relative positions of pressure catheter lumen 210, guidewire lumen 216, and balloon catheter lumen 218 may be interchanged with one another such that the pressure catheter lumen 210 may be positioned in between guidewire lumen 216 and balloon catheter lumen 218 or balloon catheter lumen 218 may be positioned between pressure catheter lumen 210 and guidewire lumen 216. Similarly, the position of guidewire lumen 216 or balloon catheter lumen 218 may be interchanged with the depicted position of pressure catheter lumen 210. As another example, the position of a balloon catheter lumen 218 may be interchanged with either pressure catheter lumen 210 or guidewire lumen 216. In this regard, one of skill in the art will appreciate that the relative positions of a pressure catheter lumen 210, guidewire lumen 216, and balloon catheter lumen 218 may be interchanged. The lumen wall 222 thicknesses (e.g., the distances between the various lumens) are representative, and not intended to indicate absolute distances or cross-sectional areas.

Referring to FIG. 2B, lumen configuration 300 may include four different lumens such as, for example, a pressure catheter lumen 310, a guidewire lumen 316, a balloon catheter lumen 318, and an injection lumen 320. Pressure catheter lumen 310 may include pressure catheter 312. Similarly, guidewire lumen 316 may include guidewire 314, which is generally sizable compared to the pressure catheter. Lumen configuration 300 presents a cross-sectional profile in which the diameter of injection lumen 320 is approximately the same size as the diameter of guidewire lumen 316, and the diameter of pressure catheter lumen 310 is approximately the same as the diameter of balloon catheter lumen 318. For example, injection lumen 320 and guidewire lumen 316 may each occupy approximately 30% of the cross-sectional area of hydrodynamic injection catheter 100, while pressure catheter lumen 310 and balloon catheter lumen 318 may each occupy approximately 15% of the cross-sectional area of hydrodynamic injection catheter 100. FIG. 2B depicts the cross-sectional area (or diameter) of injection lumen 320 as being approximately equal to the cross-sectional area (or diameter) of guidewire lumen 316. FIG. 2B also depicts the cross-sectional area (or diameter) of pressure catheter lumen 310 as being approximately equal to the cross-sectional area (or diameter) of balloon catheter lumen 318; however, the relative cross-sectional areas (or diameters) of each of these four lumens is representative and may vary between about 5% and about 10%. The relative sizes and positions of the lumens shown in FIG. 2B are representative and are not to be construed as being limiting with respect to the cross-sectional area or diameter or position of any of the four depicted lumens. For example, injection lumen 320 and a guidewire lumen 316 may be adjacent to one another relative to pressure catheter lumen 310 and balloon catheter lumen 318. The relative positions of injection lumen 320, pressure catheter lumen 310, guidewire lumen 316, and balloon catheter lumen 318 may be interchanged with one another. In this regard, one of skill in the art will appreciate that the relative positions of a pressure catheter lumen 310, guidewire lumen 316, and balloon catheter lumen 318 may be interchanged. The lumen wall 322 thicknesses (e.g., the distances between the various lumens) are representative, and not intended to indicate absolute distances or cross-sectional areas.

As shown in FIG. 2C, lumen configuration 400 may include four different lumens such as, for example, a pressure catheter lumen 410, a guidewire lumen 414, a balloon catheter lumen 418, and an contrast lumen 430. Pressure catheter lumen 410 may include pressure catheter 412. Similarly, guidewire lumen 416 may include guidewire 414. Lumen configuration 400 presents a cross-sectional profile in which guidewire lumen 420 is significantly larger than pressure catheter lumen 410, balloon catheter lumen 418, and contrast lumen 430. For example, guidewire lumen 416 may occupy approximately 50% of the cross-sectional area of hydrodynamic injection catheter 100, while pressure catheter lumen 410, contrast lumen 430, and balloon catheter lumen 418 may each occupy approximately 15% of the cross-sectional area of hydrodynamic injection catheter 100. FIG. 2C depicts the cross-sectional areas of pressure catheter lumen 410, contrast lumen 430, and balloon catheter lumen 418 as approximately equal; however, the relative cross-sectional areas of each of these four lumens is representative and may vary between about 5% and about 10%. The relative sizes and positions of the lumens shown in FIG. 2C are representative and are not to be construed as being limiting with respect to the cross-sectional area or diameter or position of any of the four depicted lumens. For example, the relative positions of pressure catheter lumen 410, contrast lumen 430, and balloon catheter lumen 418 may be interchanged with one another such that the pressure catheter lumen 410 may be positioned in between contrast lumen 430 and balloon catheter lumen 418, or balloon catheter lumen 418 may be positioned between pressure catheter lumen 410 and contrast lumen 430. Similarly, the position of contrast lumen 430 or balloon catheter lumen 418 may be interchanged with the depicted position of pressure catheter lumen 410. As another example, the position of a balloon catheter lumen 418 may be interchanged with either pressure catheter lumen 410 or contrast lumen 430. In this regard, one of skill in the art will appreciate that the relative positions of a pressure catheter lumen 410, contrast lumen 430, and balloon catheter lumen 418 may be interchanged. The lumen wall thicknesses (e.g., the distances between the various lumens) are representative, and not intended to indicate absolute distances or cross-sectional areas. In this embodiment, the DNA injection lumen may have the guidewire inserted through it, and then removed after successful localization of the catheter, with the aid of a separate contrast injection lumen. The goal in this case would be to avoid any cross-contamination of the contrast lumen and the DNA injection lumen, which could be a concern for toxicity. This catheter design may have very similar lumen arrangement to the first catheter, but now possessing separate use cases.

The techniques herein provide cross-sectional designs for hydrodynamic injection catheter 100, which vary depending on the size of the different pressure sensors and considerations of laminar flow. One example is provided in FIG. 2D, in which lumen configuration 500 may include four different lumens such as, for example, a pressure catheter lumen 510, a guidewire lumen 516, a balloon catheter lumen 518, and an injection lumen 520. Lumen configuration 500 presents a cross-sectional profile in which the injection lumen 520 is a semi-circle. Without being bound by theory, this cross-sectional design is believed to improve laminar flow of a solution during hydrodynamic injection. According to lumen configuration 500, injection lumen 520 and guidewire lumen 516 may each occupy approximately 30% of the cross-sectional area of hydrodynamic injection catheter 100, while pressure catheter lumen 510 and balloon catheter lumen 518 may each occupy approximately 15% of the cross-sectional area of hydrodynamic injection catheter 100. FIG. 2D depicts the cross-sectional area of injection lumen 520 as being approximately equal to the cross-sectional area of guidewire lumen 516, although injection lumen 520 has a semicircular profile wall guidewire lumen 516 has a circular profile. FIG. 2D also depicts the cross-sectional area (or diameter) of pressure catheter lumen 510 as being approximately equal to the cross-sectional area (or diameter) of balloon catheter lumen 518; however, the relative cross-sectional areas (or diameters) of each of these four lumens (i.e., 510, 516, 518, and 520) is representative and may vary between about 5% and about 10%. The relative sizes and positions of the lumens shown in FIG. 2D are representative and are not to be construed as being limiting with respect to the cross-sectional area or diameter or position of any of the four depicted lumens. For example, the relative positions of injection lumen 320, pressure catheter lumen 310, guidewire lumen 316, and balloon catheter lumen 318 may be interchanged with one another. In this regard, one of skill in the art will appreciate that the relative positions of a pressure catheter lumen 510 and balloon catheter lumen 518 may be interchanged. The lumen wall 522 thicknesses (e.g., the distances between the various lumens) are representative, and not intended to indicate absolute distances or cross-sectional areas.

Referring to FIG. 2E, lumen configuration 600 may include four different lumens such as, for example, a pressure catheter lumen 610, a guidewire lumen 616, a balloon catheter lumen 618, and an injection lumen 620. Lumen configuration 600 presents a cross-sectional profile in which the injection lumen 520 is an irregular semi-circle. Without being bound by theory, this irregular semi-circle design is believed to improve laminar flow of a solution during hydrodynamic injection. Similarly, lumen configuration 600 presents a cross-sectional profile in which the balloon catheter lumen 618 is also an irregular semi-circle. According to lumen configuration 600, injection lumen 620 and may occupy approximately 40% of the cross-sectional area of hydrodynamic injection catheter 100, while pressure catheter lumen 610 occupies approximately 20%, balloon catheter lumen 618 occupies about 10%, and guidewire lumen 616 occupies about 25%, respectively. The relative sizes and positions of the lumens shown in FIG. 2E are representative and are not to be construed as being limiting with respect to the cross-sectional area or diameter or position of any of the four depicted lumens. For example, the relative positions of injection lumen 620, pressure catheter lumen 610, guidewire lumen 616, and balloon catheter lumen 618 may be interchanged with one another. The lumen wall 622 thicknesses (e.g., the distances between the various lumens) are representative, and not intended to indicate absolute distances or cross-sectional areas. It is contemplated within the scope of the disclosure that the cross-section is designed to possess the minimum lumen sizes for commercial guidewires and pressure sensors that would be used with hydrodynamic injection catheter 100. The other lumens are designed then to take up the remaining space, such that the air balloon has a minimal cross-section, while the injection lumen is an irregular shape, but is maximized in cross-sectional area.

Referring to FIG. 2F, lumen configuration 700 may include four different lumens such as, for example, a pressure catheter lumen 710, a guidewire lumen 716, a balloon catheter lumen 718, and an injection lumen 720. Lumen configuration 700 presents a cross-sectional profile in which the injection lumen 720 is oval and occupies approximately 35% of the cross-sectional area of hydrodynamic injection catheter 100. Without being bound by theory, this oval design is believed to improve laminar flow of a solution during hydrodynamic injection. Similarly, lumen configuration 700 presents a cross-sectional profile in which the balloon catheter lumen 718 is an irregular semi-circle. According to lumen configuration 700, pressure catheter lumen 710 occupies approximately 20%, balloon catheter lumen 718 occupies about 10%, and guidewire lumen 716 occupies about 25%, of the cross-sectional area of hydrodynamic injection catheter 100, respectively. The relative sizes and positions of the lumens shown in FIG. 2F are representative and are not to be construed as being limiting with respect to the cross-sectional area or diameter or position of any of the four depicted lumens. For example, the relative positions of injection lumen 720, pressure catheter lumen 710, guidewire lumen 716, and balloon catheter lumen 718 may be interchanged with one another. The lumen wall 722 thicknesses (e.g., the distances between the various lumens) are representative, and not intended to indicate absolute distances or cross-sectional areas.

Referring to FIG. 2G, lumen configuration 800 may include four different lumens such as, for example, a pressure catheter lumen 810, a contrast lumen 830, a balloon catheter lumen 818, and an injection lumen 820. Lumen configuration 800 presents a cross-sectional profile in which the injection lumen 820 is oval and occupies approximately 45% of the cross-sectional area of hydrodynamic injection catheter 100. Without being bound by theory, this oval design is believed to improve laminar flow of a solution during hydrodynamic injection. Similarly, lumen configuration 800 presents a cross-sectional profile in which the balloon catheter lumen 818 is an irregular semi-circle. According to lumen configuration 800, pressure catheter lumen 810 occupies approximately 20%, contrast lumen 830 occupies approximately 20%, and balloon catheter lumen 818 occupies about 10% of the cross-sectional area of hydrodynamic injection catheter 100, respectively. The relative sizes and positions of the lumens shown in FIG. 2G are representative and are not to be construed as being limiting with respect to the cross-sectional area or diameter or position of any of the four depicted lumens. For example, the relative positions of injection lumen 820, pressure catheter lumen 810, contrast lumen 830, and balloon catheter lumen 818 may be interchanged with one another. The lumen wall 822 thicknesses (e.g., the distances between the various lumens) are representative, and not intended to indicate absolute distances or cross-sectional areas.

Referring to FIG. 2A, pressure catheter lumen 210 may have a diameter of at most about 0.35 mm. Advantageously, this lumen size a is compatible with the majority of wire-based pressure catheters available on the market today, such as those from FISO Corporation (Quebec City, Canada) (https://fiso.com/en/). The FISO pressure sensor has a Pressure sensor, FOP-M260, which is 260 microns in diameter. These pressure catheters sense pressure at their distal tip and transmit signals through electronic or fiberoptic signals to an electronic receiver system at the proximal end, which can be fed into an electronic device and interpreted. Exemplary pressure catheter lumen 210 would ideally fit relatively tightly around the pressure catheter 212 in order to avoid any additional fluid leaking in through this channel during the hydrodynamic injection. Additional sealing could be inserted into the lumen in order to close the space between the lumen walls and the pressure catheter itself as necessary. The diameter of the pressure catheter lumen 210 may range up to about 300-400 microns and would fit the majority of pressure sensor devices on the market in order to maintain user flexibility and choice. It is important for the lumen to have some room between the catheter and lumen walls in order to allow for easy insertion of the catheter through this lumen, as well as easy sliding of the catheter through the lumen, so it's position can be adjusted within the lumen as necessary, and even for the catheter to be advanced and exit the distal end of the catheter. Of note, if the pressure catheter 212 will be advanced out of the lumen into the vessel space just before hydrodynamic injection, it may reside safely within the catheter during catheter placement in the tissue of interest. In some embodiments, the pressure catheter can be inserted through the lumen after the catheters is already positioned in the designated vessel or space inside the body.

In other embodiments of the invention, the pressure sensor 212 is already placed within the catheter lumen during the initial manufacturing. This allows the user the flexibility of already having the catheter inside and not having to buy a second device. This also lowers the risk of damaging the pressure catheter during placement into the hydrodynamic injection catheter 100. In exemplary embodiments, however, the pressure catheter may still retain flexibility inside the pressure catheter lumen in order for it to be optionally advanced into the vessel space. In exemplary embodiments, the pressure catheter may be embedded into the device during manufacturing and is preferably stable and flexible in order to withstand the torsion from the catheter packaging.

Still referring to FIG. 2A, the guidewire lumen 216 may have a diameter ranging from about 0.018 inches (in order to fit the smallest guidewires) to about 0.040 inches (in order to fit the majority of guidewire sizes on the market) so as to allow guidewire 214 to be easily inserted through the hydrodynamic injection catheter 100. In some embodiments of hydrodynamic injection catheter 100, the guidewire lumen 216 may also be used to inject nucleic acid and/or protein solution, so that this lumen will effectively function as both a guidewire lumen and an injection lumen. In these embodiments, the guidewire is removed prior to injection. Given the use of the same lumen for injection of the nucleic acid solution, the designated diameter of the guidewire lumen would be much larger than the comparable diameter of the guidewire itself used for catheter placement. In certain embodiments, this dual function lumen may be circular or oval in shape and have a diameter up to 1.25 millimeters. In other embodiments, the shape of the lumen may be oblong or irregular in nature, simply filling all the excess space within the catheter that is not taken up by other lumens within the quadruple lumen catheter.

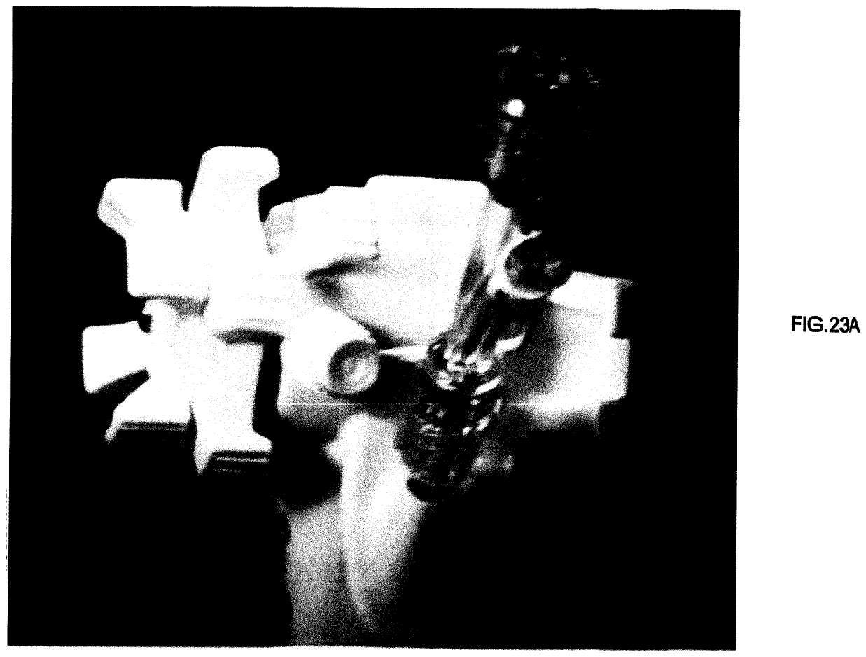

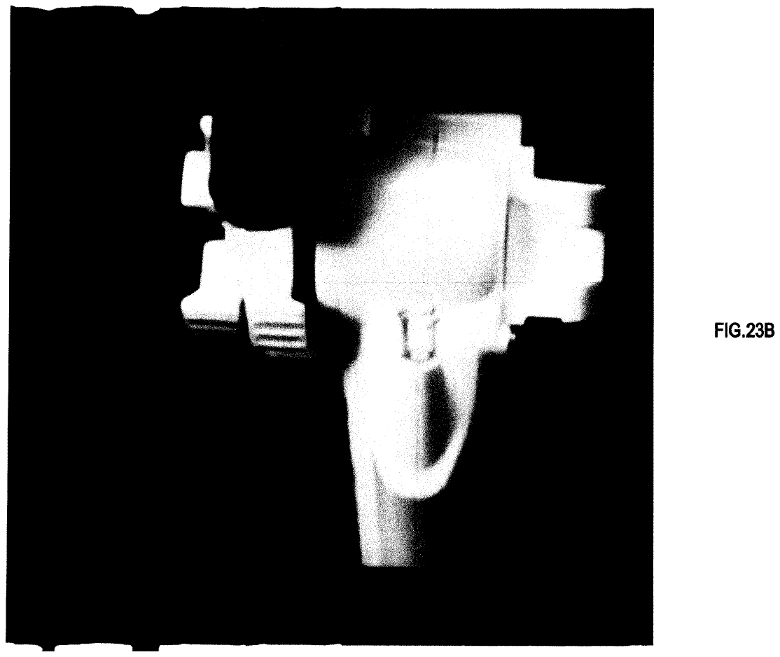

In exemplary embodiments, the guidewire 214 may be kept in during the hydrodynamic injection. The purpose of keeping in the guidewire 214 may be to assure that the catheter does not move during the injection and also assure that the catheter does not move between the period from guidewire to transition the injection. Keeping the guidewire in, furthermore, helps the user to identify specific branches of the target tissue for injection that may otherwise not be easily observe with simple contrast injection during fluoroscopic imaging. An example of this use of a guidewire for localization is in localizing the right or left hepatic ducts during a liver focused injection. For embodiments where the guidewire is left in during the injection, the guidewire lumen may be closer to the minimal size to surround the guidewire itself, wherein it merely encapsulates the diameter around the guidewire itself with minimal excess space. Moreover, keeping the guidewire in place during the injection creates a seal around this individual port, so that fluid does not regurgitate through that lumen during the hydrodynamic injection. In certain uses of the catheter, wherein the guidewire isn't kept in, but a minimal port size is kept, the guidewire lumen may be sealed off with a cap and/or syringe over a connector such as a Luer lock in order to create a pressure seal at the proximal end (FIGS. 23A-23B).

For the contrast/DNA injection lumen, the exact diameter and dimensions can be variable, since only solutions are injected through this port. In many embodiments, this lumen will be the largest lumen in the catheter, since it will accommodate the nucleic acid and/or protein injection. As stated above, increasing the diameter of the lumen that receives the hydrodynamic injection decreases the wall stress during the injection. This will allow for higher flow rates to be achieved in the catheter without causing and higher pressures to be achieved within the target tissue. Moreover, such design also decreases the expenses of the materials needed for catheter constructions, since plastics with less tensile strength may be used for building the catheter. In certain embodiments, the contrast/DNA injection lumen may be oval or circular in diameter, and is optimally 2, 3, 4, 5, or 6 French in diameter. In preferred embodiments, this lumen will be circular or oval in diameter in order to avoid fluid turbulence inside the lumen during hydrodynamic injection and ensure laminar flow. Any corners in the cross-section from a non-round shape are susceptible to lower effective flow rates from pooling of injection solution, thereby limiting the cross-sectional area that achieves laminar flow.

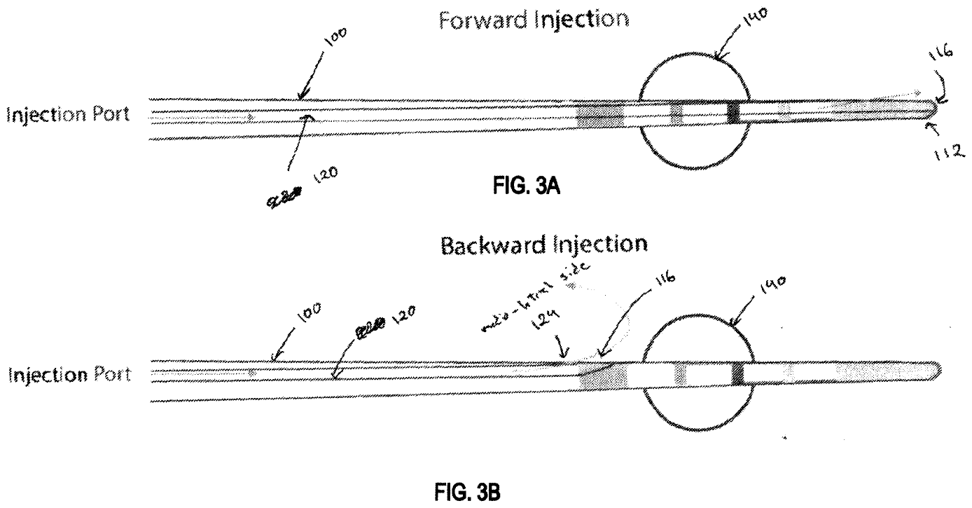

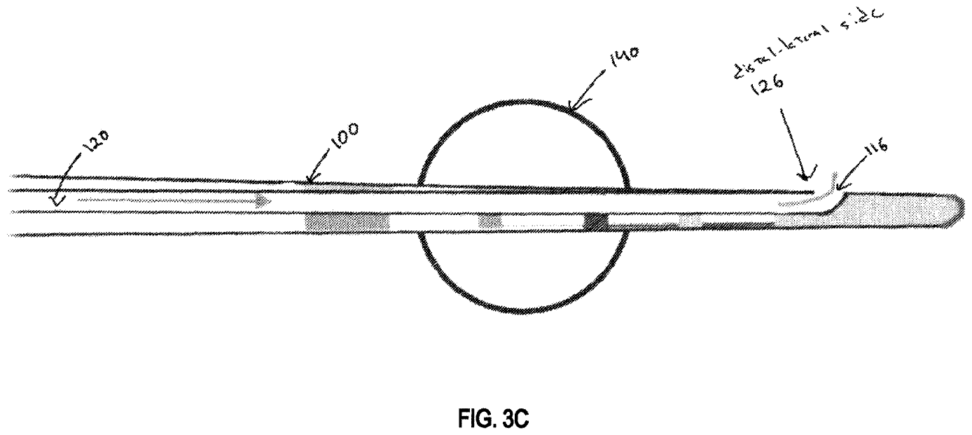

The contrast/DNA lumen is unique in the hydrodynamic injection catheter 100 design, since it has two potential openings depending on the intended anatomical use of the catheter. Referring to FIG. 3A, the injection lumen 120 of hydrodynamic injection catheter 100 may inject a nucleotide or protein solution through injection lumen 120 in the forward direction through injection orifice 116 positioned on distal tip 112, consistent with the intended direction of fluid flow so that the solution is released distal from balloon 140. As shown in FIG. 3B, the injection lumen 120 of hydrodynamic injection catheter 100 may inject a nucleotide or protein solution through injection lumen 120 in the forward direction through injection orifice 116 positioned on medio-lateral side 124 so that the solution is released proximal to balloon 140. This embodiment may be used in particular anatomical setting where there is not a direct wall along the distal portion of the catheter. As shown in FIG. 3C, the injection lumen 120 of hydrodynamic injection catheter 100 may inject a nucleotide or protein solution through injection lumen 120 in the forward direction through injection orifice 116 positioned on distal-lateral side 126 so that the solution is released distal to balloon 140 but not at distal tip 112. This embodiment may be used in particular anatomical setting such as the kidney pelvis, wherein there is not a direct wall along the tip aspect of the catheter.

According to the techniques herein, this multi-lumen catheter system for hydrodynamic gene delivery may be optimized for percutaneous injection into the biliary system. In this system, ultrasound, CT, or MRI is used to first to place a needle through the skin and into the targeted duct, vessel, or space. Subsequently, the sheath is inserted into the duct, vessel, or space, followed by the catheter being advanced through the sheath in order to enter that same space. In certain embodiments, the sheath contains a balloon that can be inflated at its tip, thereby blocking flow of solution through the entry site into the duct, vessel, or space. The sheath can also optimally contain radio-opaque material to confirm localization. The gene delivery catheter design used with the sheath and percutaneous routes for the liver is optimally designed to have an injection lumen opening proximal to the balloon, such that the fluid will still go toward the liver, even as the catheter is being localized away from the liver. This is illustrated in FIGS. 3A-3B.

According to the techniques herein, the catheter's contrast/DNA lumen will exit along a lateral aspect proximal to the balloon. In these applications, the inflated balloon would be upstream of the fluid exit, causing flow in the proximal direction toward the user during hydrodynamic injection. The intended use of this catheter is for situations where the catheter is inserted into a vessel in the upstream region of the tissue, where one would want the injection to occur. An example of this would be introducing the catheter into the biliary system through percutaneous skin entry directly into the liver guided by imaging equipment such as ultrasound. This is opposed to more common situations such as in ERCP where the catheter is inserted into the downstream trunk of a vessel system, in this case the biliary system, and the fluid is pushed in a retrograde fashion in the upstream branches of that vessel system.

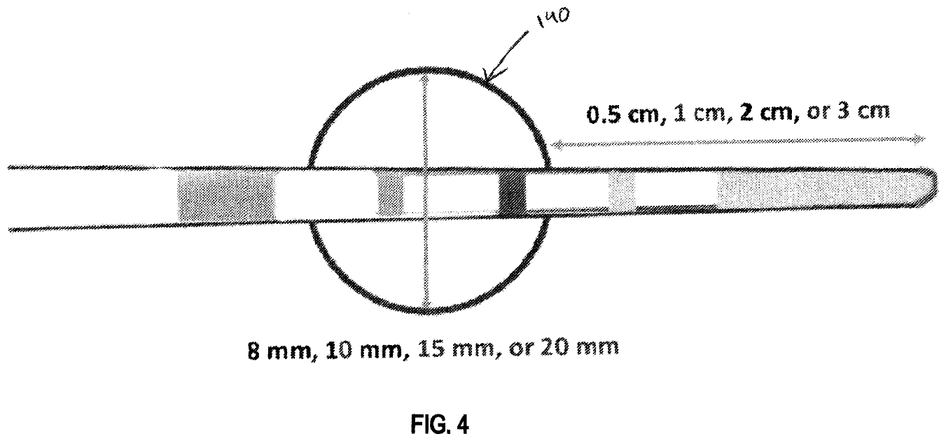

The fourth lumen of the quadruple lumen catheter is the lumen for the air that will eventually inflate the balloon that creates the seal during fluid injection. In this lumen, air is simply communicated from an air syringe to the balloon at a designated distance from the end of the catheter. At the proximal end of the lumen is a connector such as a Luer lock that connects to air syringe. The syringe would settings of how many milliliters of air to inject in order to fill out the balloon to a given diameter. The design and use of this syringe/balloon inflation system is extensively covered in another catheter patents in the art. As shown in FIG. 4, the balloon distance from the tip of the catheter may be at least about 1 cm, about 2 cm, or about 3 cm in certain embodiments. This gives it extra room in order to invade certain tissue spaces and assures that the solution exit is well past the balloon area, thereby reducing pressure on the balloon.

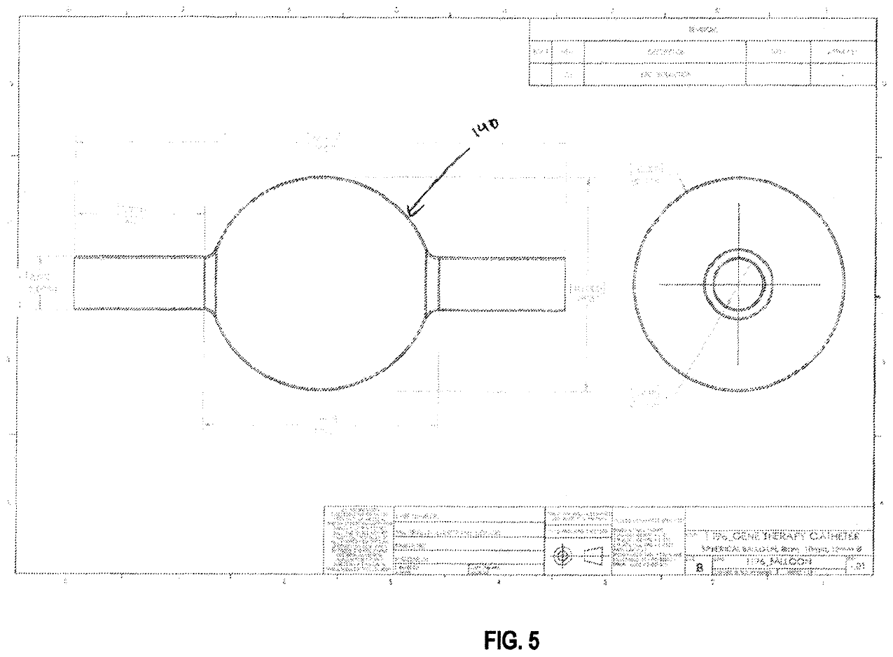

Alternatively, in other preferred embodiments, the balloon is less than 1 cm from the tip of the catheter, or less than 0.5 cm, in order to avoid missing any vessel branches from the catheter tip being too long (see e.g., FIG. 4). In this embodiment, the distal tip 112 of the hydrodynamic injection catheter 100 would be short and stationary within the center of the balloon. The balloon 140 in some embodiments could be circular shaped or alternatively with cylindrical shaped. The balloon 140 in these embodiments would serve to center the catheter tip in the center of the vessel or duct, such that fluid would exit in the forward direction and not press on any catheter walls. An example of the structure of balloon 140 is provided in FIG. 5.

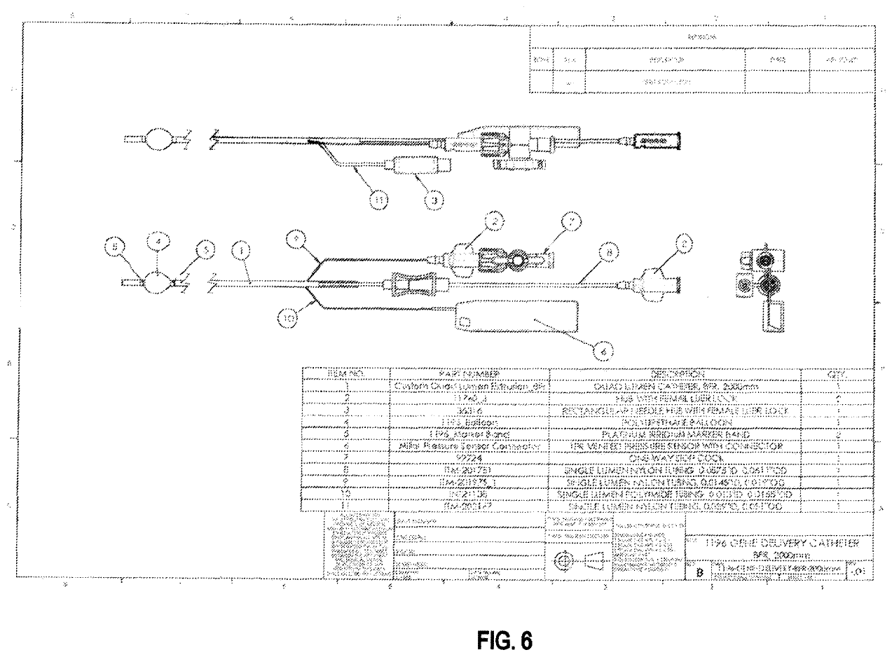

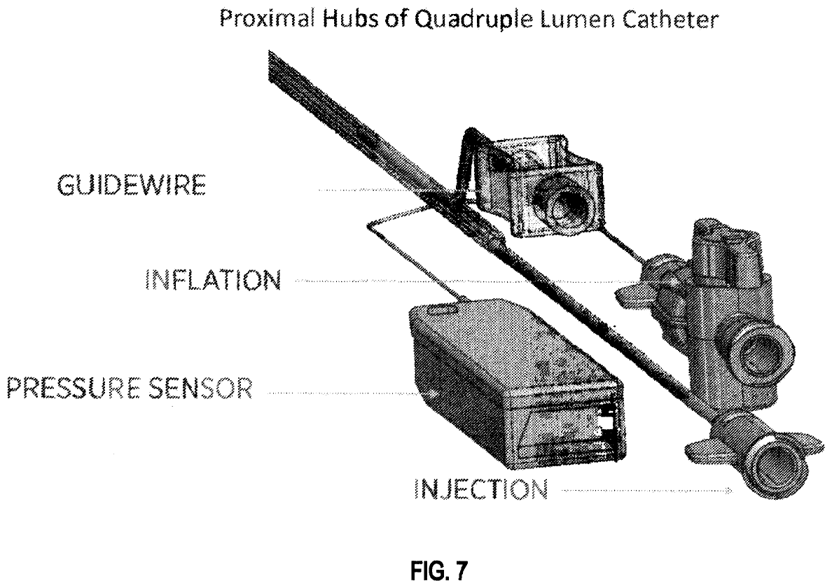

An exemplary depiction of all components of an exemplary catheter design are depicted in FIG. 6, covering the proximal components that the user interacts with, as well as the distal tip design with the balloon. Artist renderings of the proximal hub of the catheter are depicted in FIG. 7.

Another alternative gene delivery catheter has different features and utility. In this quintuple port, quadruple catheter design, the goal is to have two different injection ports. The first injection lumen releases fluid downstream of the balloon, and the second injection lumen releases fluid upstream of the balloon. The purpose of this design is to verify the position of the catheter within a vessel or ductal system. Specifically, vessels often have several branches, and the appropriate injection point is at a specific point in this system. An example is the location of the catheter within the biliary system. The optimal location of the catheter is within the common hepatic duct, which is an extension of the common bile duct extending past the cystic duct that connects to the gallbladder. If hydrodynamic injection were to proceed in the common bile duct, then fluid would also be injected into the gallbladder, which has a sizable volume. Fluid would not accumulate in the liver and the pressure achieved would be minimal and insufficient to deliver DNA inside cells.

During an endoscopy or interventional radiology procedure, the catheter is advanced, and contrast is serially injected to elucidate the branching network. Once the appropriate catheter position is reached, it is possible for the catheter to slip out of position. This can be particularly challenging for placement in the common hepatic duct, wherein the cystic duct branch can be close to the bifurcation of the right and left hepatic branches, leaving only a minimal length of the common hepatic duct for positioning. Furthermore, it is possible for the balloon to slip backwards during injection itself, thereby unknowingly introducing fluid into the gallbladder. This situation could be avoided by having a second contrast injection lumen on the opposite side of the balloon. This would allow contrast to be injected on both slides of an inflated balloon, thereby visualizing the precise position of the catheter and balloon within the entire vessel or ductal network.

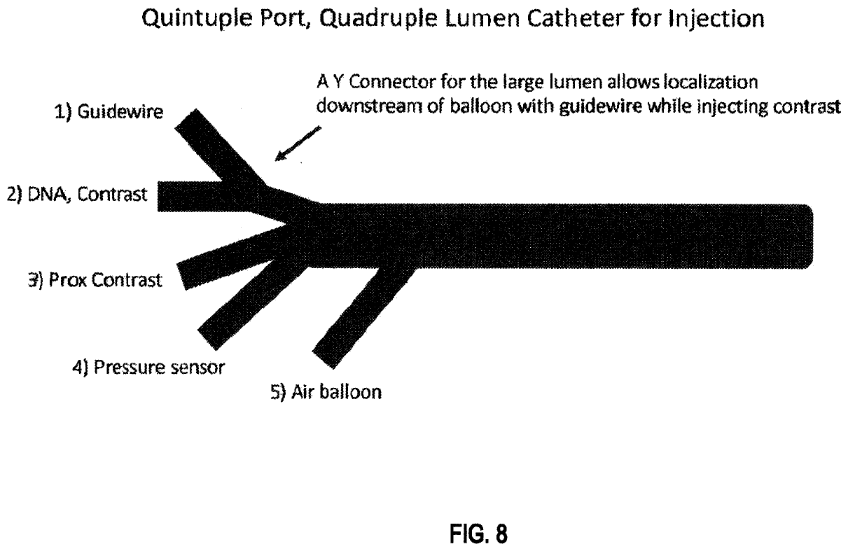

In order to achieve this goal, a new catheter design is outlined. The catheter user end is depicted in FIG. 8, showing the quintuple port design. The cross-sectional design for the catheter is depicted in FIG. 2G. This catheter has one lumen for air to inflate the balloon. A second lumen contains the pressure catheter to monitor the injection in real time. A third lumen would be utilized to inject contrast through the catheter with an opening on the side wall proximal to the balloon. This would be used to identify upstream branches of a vessel or ductal network that the balloon is now distal. The third lumen would be smaller in diameter than the fourth lumen, since it would not need to receive fluid injection at high pressure. The fourth lumen would be larger in diameter than the third lumen and serve to inject contrast at downstream vessels to the catheter tip. This lumen would be forward facing and also serve to deliver DNA solution in the forward direction.

Another embodiment of the gene therapy catheter would include only three lumens through the catheter itself, but four functional lumens at the user interface/handle. For this configuration, the largest lumen would serve for both guidewire and DNA/contrast injection. This lumen would be connected to a port at the proximal user handle that is attached to a Y-shaped adaptor (FIGS. 23A-23B). This Y-adaptor would allow for the entry to the guidewire on one side, while the other side serves to allow for simultaneous contrast injection through the same lumen. Given that contrast is only slowly injected by hand through this lumen, the simultaneous presence of the guidewire and contrast solution is expected to offer minimal interference.

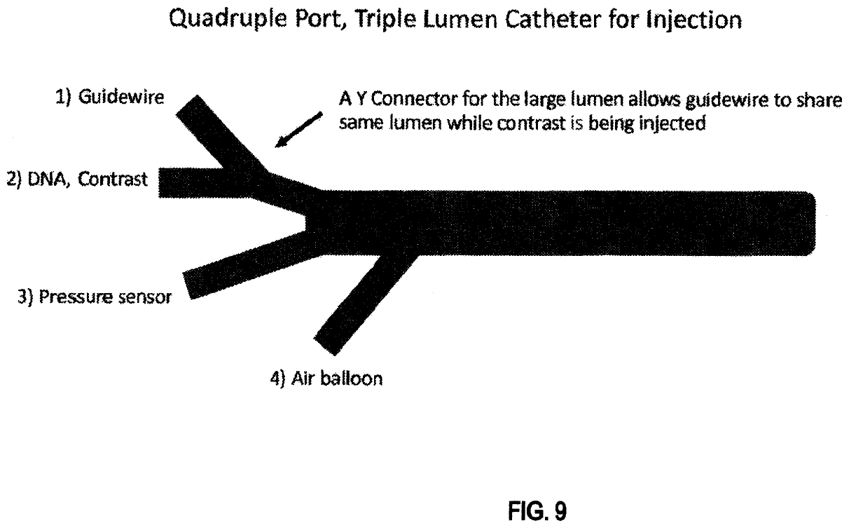

To inject DNA, the Y-adaptor could be removed, or alternatively kept in during the injection. The unused guidewire port during the injection would be sealed with a cap such that no fluid would leak from it. In another embodiment, the Y adapter is a physical part of that particular port such that it is non-removable. The advantage of this design is that the largest possible injection lumen can be designed, which is useful for mediating the highest flow rates and pressure through the catheter possible for hydrodynamic injection. A representation of the catheter user end is depicted in FIG. 9.

One embodiment of the gene therapy catheter would be a simpler version of the current triple lumen catheter, wherein the current catheter is modified to have the two lumens be forward facing, instead of the current Multi-3 V Plus Olympus catheter, which only has one forward facing lumen. One lumen would be used for the guidewire or pressure catheter and be smaller in size, being at least 0.040 inches in diameter. The other lumen is equivalent in size or larger, and which serves for injection of contrast or DNA solution. Both lumens are optimally circular or oval in shape. The cross-sectional of both lumens would take up the majority of the area within the cross-section. The air lumen could be variable shapes, including triangular, in case to accommodate the other two lumens.

The optimal diameter of the catheter is defined by the clinical need and use case. For example, the diameter of the human bile duct is 4 mm on average but can be dilated up to 6-8 mm in size in some individuals. On the opposite spectrum, some patient bile ducts can only be 2-3 mm in diameter. The pancreatic duct is 3.5 mm in the head of the pancreas, and 2.5 mm in the tail of the pancreas, and the ureter is 6 to 8 mm.

The working channels of the ED-580XT Duodenoscope (Fujifilm) for ERCP is 4.2 mm, representing a standard duodenoscope that would be used among commercial providers. An example of the working channel for a commercial cystoscope, CYF-V2/VA2, (Olympus Med) is 2.2 mm.

The catheter for hydrodynamic injection should be able to fit within any working channel or percutaneous IR needle as necessary in order to access the body as a minimum requirement. While the diameter could be contemplated to be very close to the diameter of the working channel, there is an advantage to having the catheter be smaller for maneuverability. Furthermore, a small catheter could avoid trauma on individuals with smaller bile ducts, including infants and neonates.

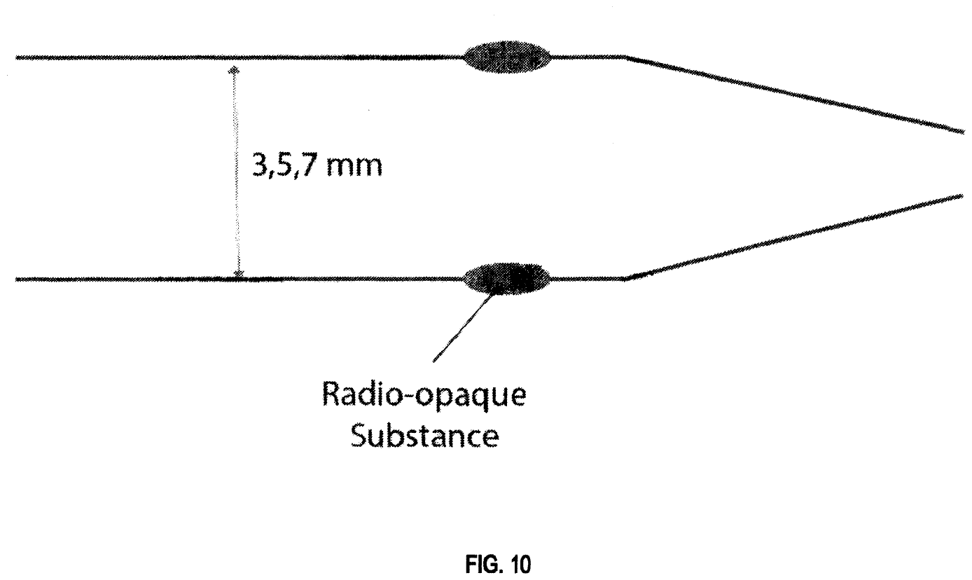

In preferred embodiments, the outer diameter is 8 Fr in order to fit in bile ducts and the working channel of the endoscope easily. In other embodiments, the outer diameter is a maximum of 9 Fr or 10 Fr in size (see e.g., FIG. 10). For renal applications, the catheter may be more optimally 5 Fr, 6 Fr, or 7 Fr in diameter (see e.g., FIG. 10). These smaller sizes are also advantageous in being versatile for both biliary and ureter ducts.

In certain embodiments, the catheter has an outer diameter of 2.5 mm (7 Fr) in size and is able to be used for renal and liver/pancreas applications.

As an example, the Multi-3 V Plus Olympus catheter currently used in experiments has a distal tip outer diameter of 4.5 Fr and a minimum working channel size requirement of 2.8 mm. Thus, a potential diameter size for the gene therapy catheter could be at least this size in certain embodiments.

The catheter is optimally 190-200 cm in total length, in order to fulfill doing ERCP procedures, reaching from the mouth all the way into the biliary system of the liver. In certain preferred embodiments, the catheter is 195 cm in length. For other applications in other organs, the catheter length could be reduced to match the physical needs of the procedure. Alternatively, the excess length of the catheter could simply not be used and remain outside of the body. This latter option would be advantageous due to simplifying manufacturing with one device able to service gene delivery procedures in all organs.

A crucial aspect for the use of these gene therapy catheters for hydrodynamic injection is the monitoring of pressure during the procedure in order to assure successful injection. As outlined in the different catheter embodiments above, a dedicated lumen for the pressure sensor will be installed. In preferred embodiments, a commercial pressure sensor will be insert it through this dedicated lumen, either by the user or pre-packaged when the catheter is sold. A key aspect of the catheter will be where to place the exact pressure sensor in order to get accurate readings of the pressure in the vessel space.

The ability to place the pressure sensor outside of the catheter such that it would be lodged directly into the vessel or duct has been assessed. It would be logical to understand this might accurately measure the pressure being felt along the vessel or duct walls. However, this location has two significant disadvantages. The first is advantage is that it requires the pressure sensor to be mobile by the user since the catheter can it be inserted into the scope and docs with the pressure sensor tip outside of it. The pressure sensor tip would be readily damaged, such that the catheter and pressure sensing would be rendered useless. The other major challenge is that it's possible that when the pressure sensor is placed outside of the catheter, that its proximity to the injection lumen and channel is such that the pressure being felt is exclusively from the flow rate, and not the steady state pressure being experienced by the vessel or duct.

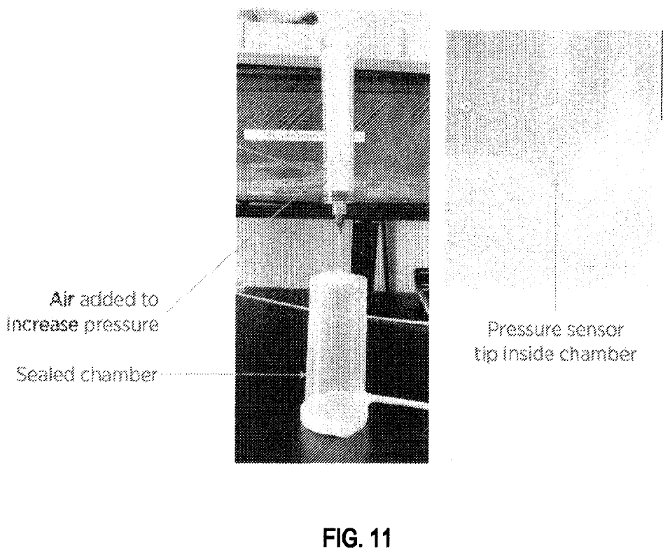

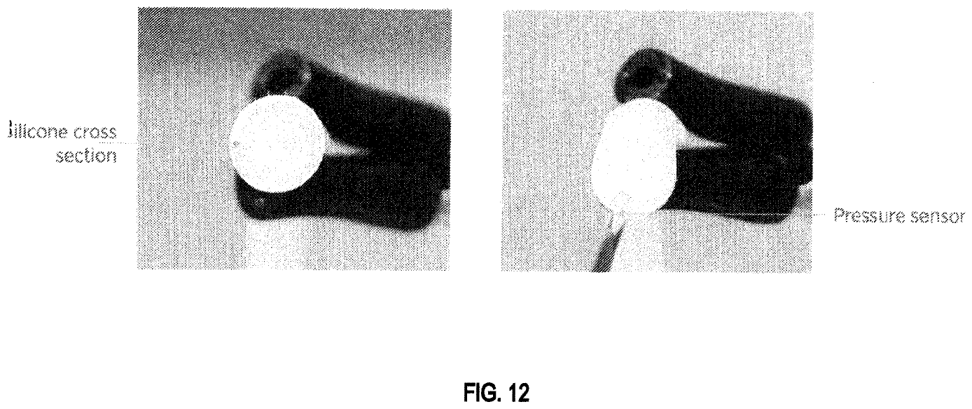

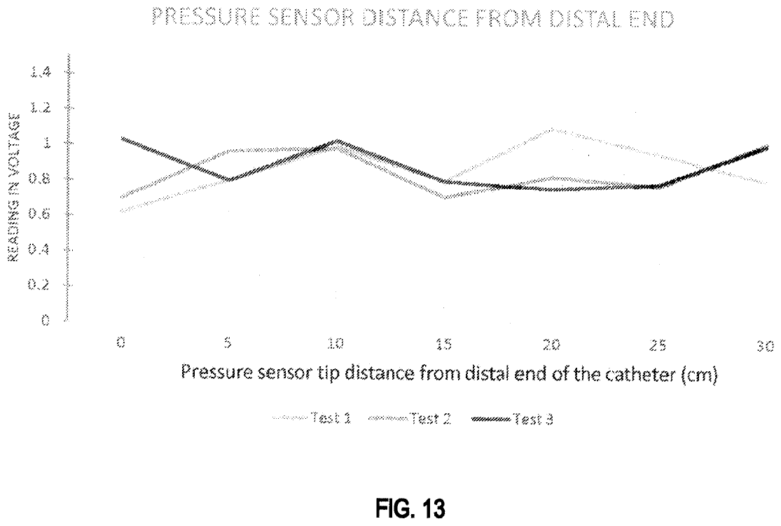

In order to address these challenges, the current technology makes several improvements based on non-obvious discoveries. The first discovery is that the pressure readings during injection for a pressure sensor located just outside the catheter versus a pressure sensor just inside the catheter were equivalent. This was observed during benchtop testing of a pressure sensor in a catheter, which is summarized in FIG. 11 and FIG. 12. Investigations observed pulling the catheter back to 30 cm still results in the same pressure observed as at the tip of the catheter (see e.g., FIG. 13). This suggests that the pressure read by the pressure sensor when located outside the catheter was actually genuine hydrostatic pressure. This also points to the fact that the fluid pressure inside the vessel or duct feeds its way back into the open lumens of the catheter such that the pressure felt inside that channel or lumen is contiguous with the outside.

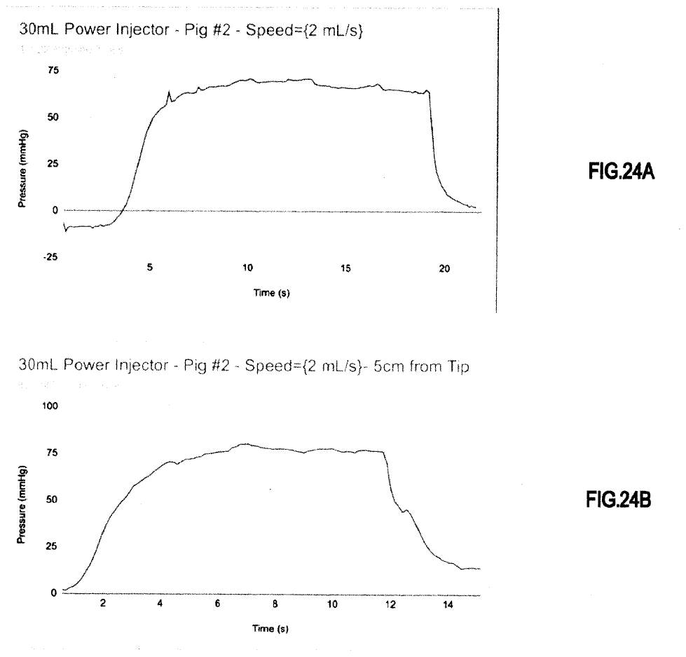

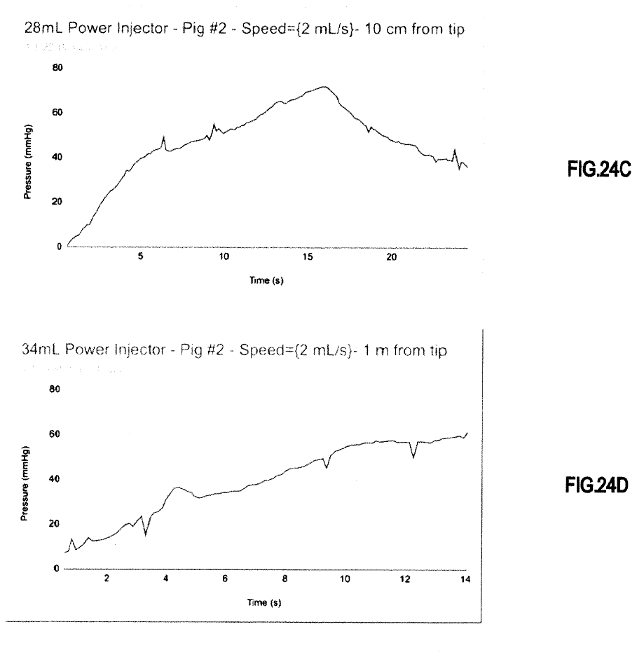

This discovery has several beneficial aspects to it, since the pressure sensor can now be better protected inside the catheter without the need to adjust its position during the procedure. While the benchtop testing was promising, it was still uncertain how it would respond to the authentic in vivo environment within the pig. Moreover, the question arises how far the pressure sensor can be located within the catheter lumen, but still yield an accurate reading. We did a series of testing for this question during ERCP with hydrodynamic injection of set fluid volumes and flow rates with a power injector, placing the catheter at shorter intervals to the catheter tip, and then dramatically longer intervals (FIGS. 24A-24D).

Pressure readings up to 50 cm inside the catheter from the tip were similar to the outside of the catheter were observed. At distances 100 cm and 150 cm from the catheter tip, as well as when the pressure tip sensor was placed at the handle, the pressure reading was delayed in reading and/or inaccurate compared to other pressure readings at different locations at a given flow rate. Thus, optimal placement of the pressure catheter for this invention would be 50 cm or closer to the tip of the pressure catheter. This discovery also carries the added benefit of allowing shorter pressure sensors to be utilized, which allows for a range of sizes among available pressure catheters to be utilized.

The pressure sensor lumen can either be forward-facing or side facing at the tip depending on the discretion of the user. The benefit of forward-facing is easy communication with the external fluid environment being injected, along with simplicity of manufacturing. The benefit of a side-facing port is that it would be less susceptible to turbulent flow during the injection, such that the pressure measured may be more accurate to the equilibrated external fluid pressure. To gauge against the potential for fluid turbulence for the forward-facing modality, the pressure sensor could be retracted back into the catheter a minimal distance.

In certain embodiments, wherein the pressure sensor does extend to the tip of the catheter. The pressure sensor in these embodiments is 195 cm in length.

The inflation size of the balloon of the catheter is alternately variable matching the potentially diverse vessels that the catheter could be inserted into. It is envisioned that the catheter could be maximally inflated to 10 millimeters, 15 millimeters, or 20 millimeters in diameter which would match maximal distances in vessels or spaces to seal off pressure. For example, the diameter of the human bile duct is 4 mm, the pancreatic duct is 3.5 mm, and the ureter is 6-8 mm. Minimal balloon inflation diameters include 5 millimeters or 8 millimeters to accommodate smaller vessel sizes. Examples of the balloon structure and design are provided in FIG. 5.

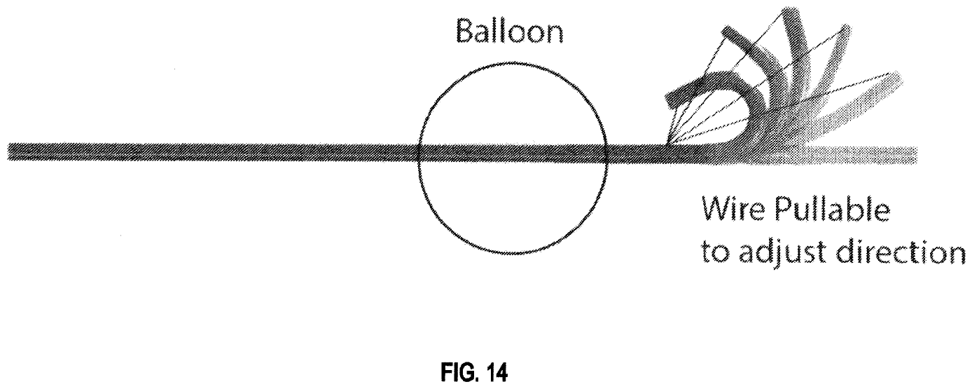

In some embodiments, a catheter will be modified to have a steerable tip. The steerable feature is a common component in the design and construction of other catheters, wherein a wire pulls tension on the catheter tip, such that the tension can be controlled by handle, lever, or clamp at the proximal catheter end by the user (see e.g., FIG. 14). In many clinical applications today, different catheters are used during procedures, such that one catheter has the steerable tip to localize the catheter into the correct vessel, often steering it away from other vessels. Subsequently, the catheter can be removed over a guidewire and a second catheter driven over the same guidewire, wherein the second catheter has an alternative design. While the system is functional similar to other procedures with two catheters, the developing of a single gene injection catheter in some embodiments is simpler and more straightforward. The single catheter has both features, such that it can be steered into any orifice or vessel branch along with performing the requirements of gene delivery. In order for this steerable catheter to be designed, it is important to note that the wire device controlling the catheter tip end will pass under the balloon. The balloon would extend over its surface. This would allow for tension to still go through the system without interrupting an inflated balloon.

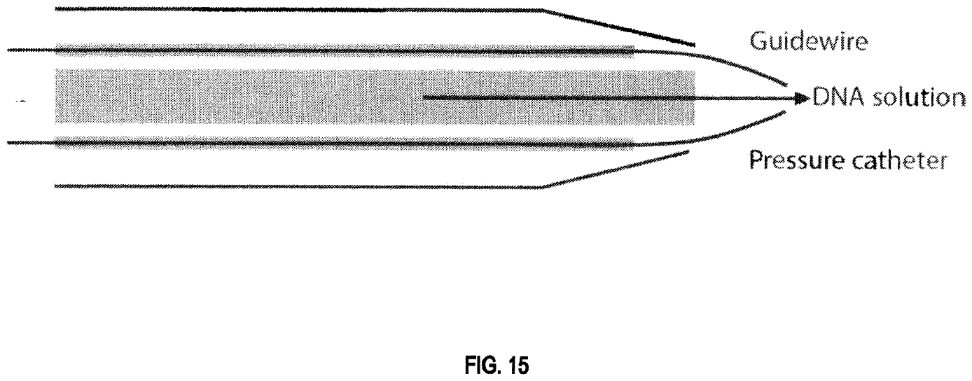

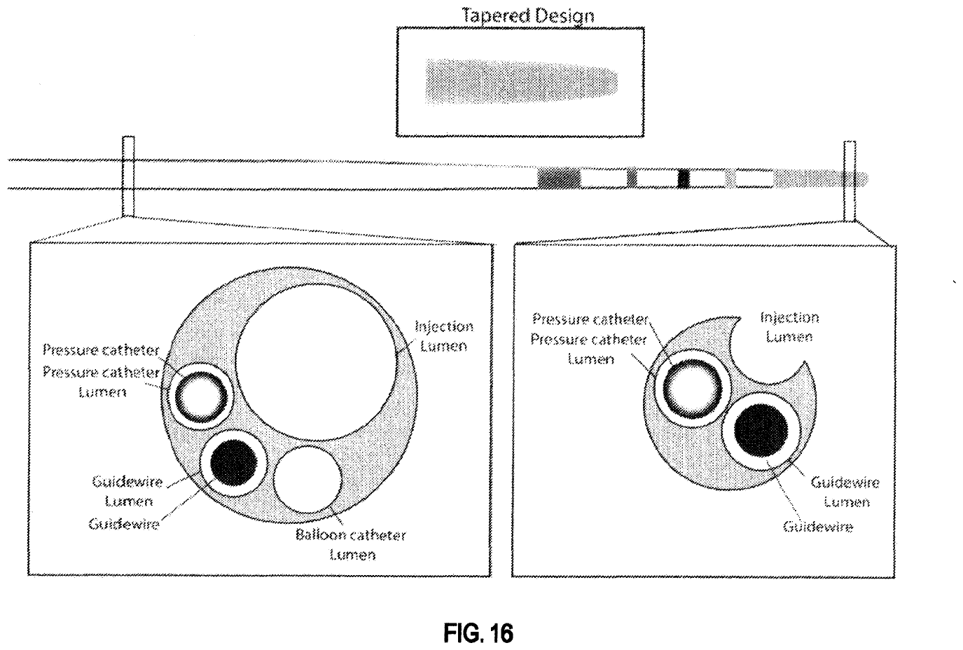

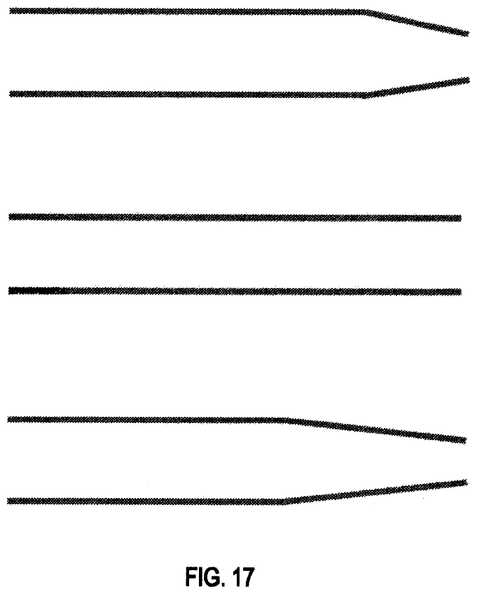

In some embodiments, the catheter will have a tapered tip in order to facilitate insertion through different anatomical orifices and help guide it into different vessels. Note that the tip tapering must still allow the guidewire, pressure sensor to exit through the catheter, in addition to not obstructing the DNA solution flow too much (see e.g., FIG. 15). In practice, however, this could be difficult, which is why in preferred embodiments, the catheter may have a blunt end such that the diameter of the tip matches the diameter throughout the rest of the catheter. A possible solution for these challenges is to have the taper go over the cross-sectional area, such that it will preferentially only decrease the injection lumen size, opening it up to the surface (see e.g., FIG. 16). For the taper at the catheter, the diameter of the catheter will be decreased at a gradual slope, before a smaller diameter finishes off the end of the catheter. The different versions of tapering exist in FIG. 17.

Concerning the materials that the catheter will be constructed from, the catheter is optimally made of plastic materials for relative softness and flexibility, but the plastic should have relatively high tensile strength in order to withstand a high pounds per square inch (psi) pressure on its walls during a hydrodynamic injection. The catheter in most embodiments should tolerate a pressure up to 1000 psi of pressure on its walls. In preferred embodiments, that catheter withstands a pressure up to 2000 psi on the catheter walls.

The gene injection catheter should include its distal tip radio-opaque material which will form a distinctive pattern to allow it to be easily visualized by different imaging mentalities including fluoroscopy during a procedure. An example of the radio-opaque marker sand design is provided in FIG. 10.

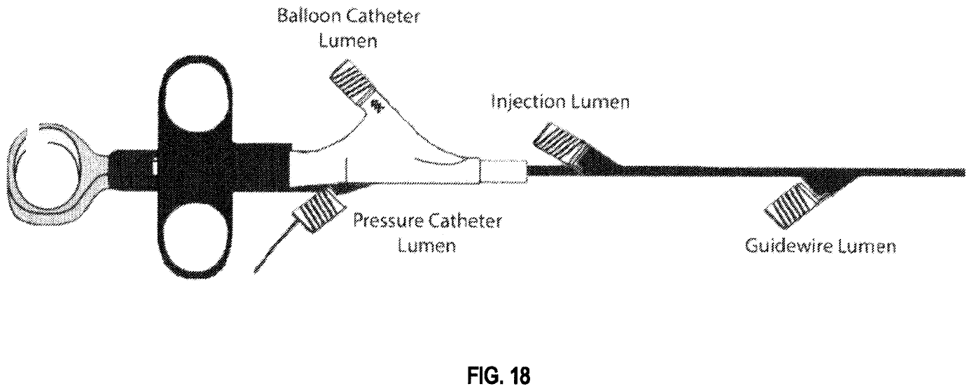

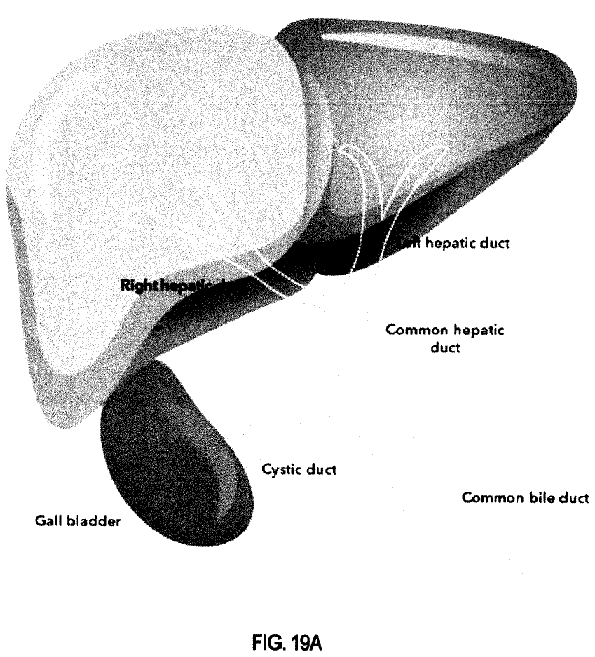

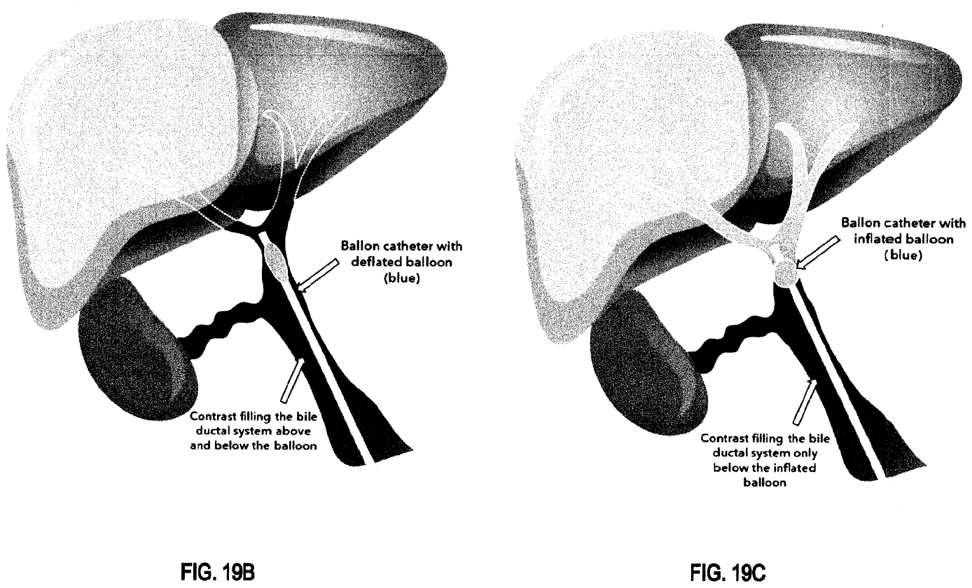

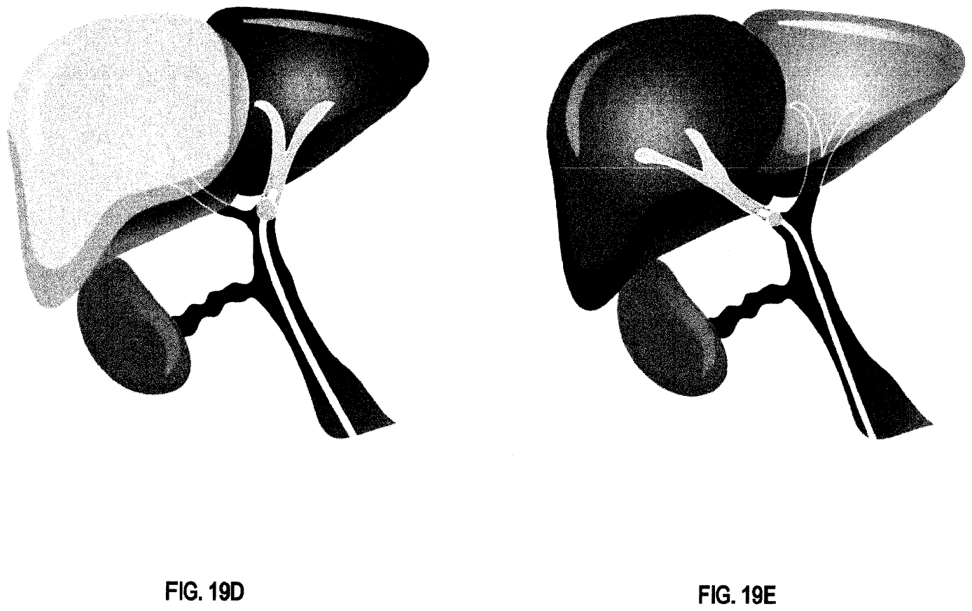

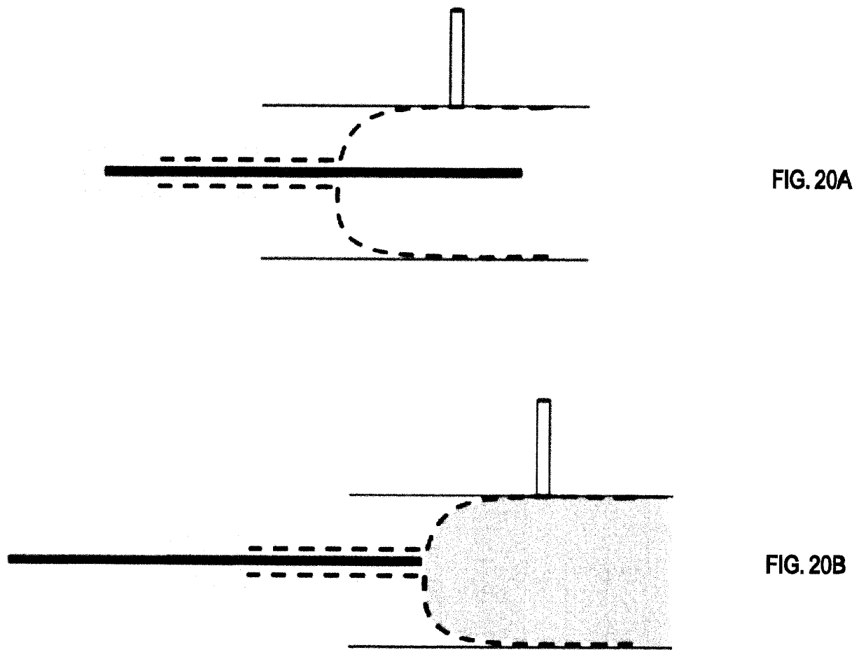

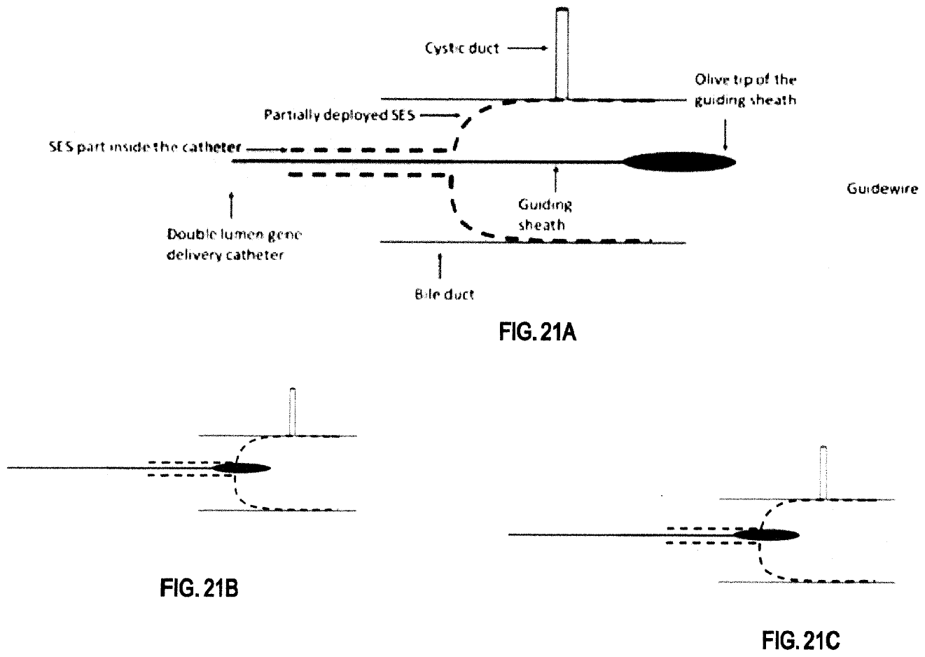

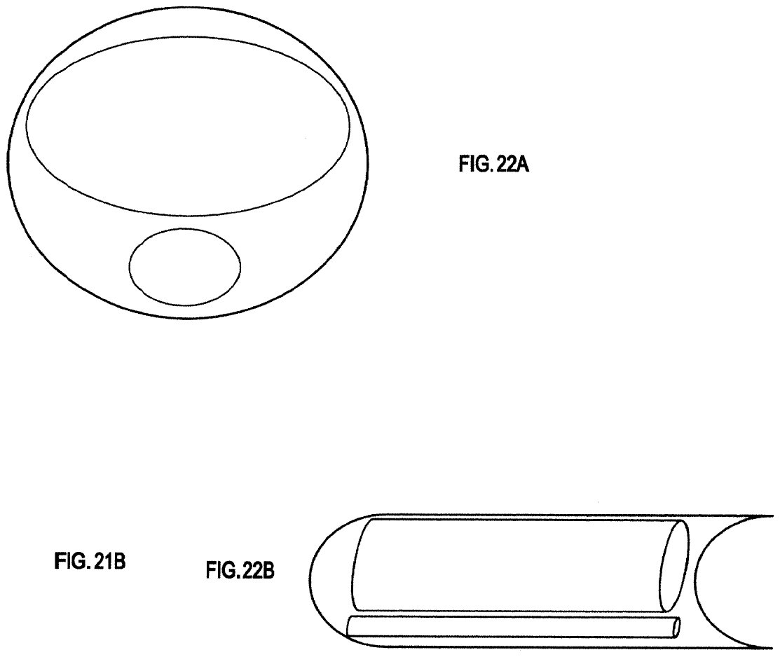

All the different lumens of the catheter should have Luer locks at their ends or ports to allow their potential sealing to prevent any leakage of fluid solution in case those specific lumens aren't being used during that procedure. The sealing of each individual lumen will increase pressure in the said lumen in order to prevent backflow of the solution. The catheter handle may have many different shapes and forms to connect to all the different lumens. One example is provided in FIG. 18.

Concerning the different use cases of the catheter, the catheter is envisioned toward injecting nucleic acid and/or protein solutions into a variety of organs. The different vessels that catheter may be inserted vary widely, but include the bile ducts, pancreatic ducts, ureters, different veins and arteries in the body, and different bronchial branches in the lungs.

In all these tissues and application areas, the catheter will be employed for the efficient injection and nucleic acid and/or protein solution, while reliably monitoring pressure during the injection and having an efficient balloon seal. The guidewire may be kept in during the injection in some embodiments in order to maintain catheter's positioning in the vessel or duct. The radio-opacity of the guidewire markings and the radio-opacity of the catheter tip will help gauge the depth of the catheter and any movement of the catheter during injection.

The catheter is intended toward use during endoscopic retrograde cholangiopancreatography, directly hydrodynamic injection into the liver or pancreas.

The catheter is intended toward use during interventional radiology-guided percutaneous access of the biliary system, being inserted through the skin directly into the biliary system. The goal would be for hydrodynamic injection into the liver.

The catheter is intended toward use during cystoscopy, wherein the catheter would be advanced into the ureters.

### References

Novel triple lumen catheter for ERCP tissue diagnosis

https://www.ncbi.nlm.nih.gov/pmc/articles/PMC6075948/pdf/10-1055-a-0591-2740.pdf

Multi-lumen catheter for performing endoscopic interventions

https://patents.google.com/patent/DE102015114538A1/en

Injection tube for catheter devices

https://patents.google.com/patent/US8029473

Dual Balloon Biliary Stone Extraction Device

https://patents.google.com/patent/US20150150572A1/en

Organ directed gene delivery

https://patents.google.com/patent/US20200345867A1/en

### Example 1. Benchtop Test Pressure Sensor Accuracy Test

The pressure sensor's ability to measure pressure when retracted from the distal tip was tested. Pressure sensor was reinforced inside 0.030″ ID Polymide tubing that represented a rudimentary catheter system.

The initial test had the pressure sensor tip inside a sealed chamber. This was documented as distance 0 cm. The pressure sensor was then retracted from 0 cm to 30 cm, 5 cm at a time. Three tests were performed at each distance.

A 10 cc syringe was used to increase pressure inside a plastic container sealed with hot glue. Air was used instead of saline to test for worst case. Air did leak around the pressure sensor inside the lumen but was a slow leak.

Pressures recorded were higher than what would be seen in a clinical setting, but the granularity of the sensor is high enough that even at lower pressures the readings were similar no matter the distance from the tip. A silicone dual lumen catheter was used to insert the pressure sensor into a sealed chamber. The pressure sensor was inserted through the small lumen and the large lumen was sealed on the distal end.

The conclusion was that no significant difference in readings as the pressure sensor is retracted from the distal tip up to 30 cm. This suggests that a shorter pressure sensor than the 200 cm catheter length could be used for pressure readings.

1. A catheter system comprising:
a catheter with a catheter body having three or four mutually independent lumens extending through the catheter body including:
(a) an optional first lumen for receiving a pressure catheter/transducer,
(b) a second lumen for receiving a guidewire,
(c) a third lumen for receiving and transmitting a solution, and
(d) a fourth lumen for transmitting air to inflate a balloon at the distal end of the catheter.

2. The catheter system of claim 1, wherein the solution comprises a nucleic acid or a protein solution.

3. The catheter system of claim 1, wherein the catheter body has a substantially cylindrical shape of substantially uniform diameter along a longitudinal axis; or
wherein the second lumen and the third lumen can be combined into a single lumen; or
wherein the combined lumen has a dual port to allow insertion of the guidewire and injection of the solution through the combined lumen; or
wherein the second lumen and the third lumen are larger than the first and fourth lumens; or
wherein the third lumen is the largest lumen in diameter, and the other three lumens are a minimal size for their functional purpose; or
wherein the third lumen has the largest diameter and allows for decreased catheter wall tension and greater flow rates during injection; or
wherein the first lumen has a diameter of less than or equal to about 300 microns, less than or equal to about 400 microns, less than or equal to about 500 microns, less than or equal to about 600 microns, less than or equal to about 700 microns, or less than or equal to about 800 microns; or
wherein the pressure sensor inserted into the first lumen is connected to external electronic system, such that real-time pressure can be monitored during the injection; or
wherein the first or second lumens have an oval or round cross-section to facilitate insertion of a substantially circular pressure sensor and guidewire; or
wherein the second lumen has a diameter larger than 0.018 inches, 0.025 inches, 0.035 inches, or 0.045 inches; or
wherein the first lumen has the pressure sensor embedded in it; or
wherein the pressure sensor is embedded and able to be advanced forward beyond the distal tip of the catheter into a duct or vessel in order to sense fluid pressure directly in the duct or vessel during injection; or
wherein the pressure sensor can be retracted inside the catheter to positions up to 50 cm away from the tip of the catheter and continue to sense fluid pressure outside of the catheter body; or
wherein the pressure sensor has a length sufficient to reach the tip of the catheter, for example 195 cm; or
wherein the pressure sensor has a length which terminates inside the catheter, for example 140 cm; or
wherein the fourth lumen has the smallest diameter among the four lumens; or
wherein the fourth lumen terminates at the proximal end at a connection configured to provide access by syringe for air injection; or
wherein the fourth lumen has a non-circular or non-oval shape to accommodate the cross-sectional areas of the other three lumens; or
wherein the balloon is located 0.5 cm, 1 cm, 2 cm, or 3 cm from the end of the catheter; or
wherein the balloon is positioned at a distance less than 1 cm from the end of the catheter; or
wherein the balloon has a maximum diameter of 8 mm, 10 mm, 15 mm, 20 mm, or 25 mm after inflation; or
wherein the catheter body has increased a tensile strength sufficient to avoid breaks and/or tears, tolerates flow rates between 2-20 mL/sec, and withstands pressures between 500-2000 psi; or
wherein the first three lumens have forward facing exits at the distal end of the catheter; or
wherein the second and third lumens are forward facing, with the first lumen lateral facing and positioned distal to the balloon; or
wherein the distal tip of the catheter is tapered with a smaller diameter than the diameter of the major length of the catheter; or
wherein the diameter of the taper is greater than the diameter of the guidewire, the third lumen, and the pressure catheter combined, allowing the guidewire and pressure catheter to exit the distal tip of the catheter; or
wherein the distal tip of the catheter is not tapered with all lumens forward facing in consistent size; or
wherein the total diameter of the catheter is 4.5 French, 5 French, 6 French, 7 French, 8 French, 9 French, or 10 French in size; or
wherein all lumens include connectors at their proximate ends configured to allow for connection of other lines/devices for the injection of fluid; or
wherein the distal tip of the catheter is steerable with a tension wire; or
wherein the tension wire extends no further than the balloon; or
wherein the third lumen has a lateral opening at a position proximate to the air balloon; or
wherein the distal tip of the catheter is coated in a radio-opaque substance; or
wherein the balloon is coated in a radio-opaque substance; or
wherein the catheter is configured to be passed through a catheter sheath, optionally further comprising the catheter sheath; or
wherein the catheter sheath comprises an inflatable balloon at its tip, wherein after inflation flow of any solution proximate to the inflatable balloon is blocked; or
wherein the catheter sheath contains a radio-opaque material, optionally further comprising an adaptor around the guidewire and/or pressure catheter to seal the respective lumen and prevent fluid leak; or further comprising an additional third lumen, one third lumen having a lateral opening proximate to the balloon and the other having a forward-facing opening at the distal end; or further comprising an additional first lumen with an opening proximate to the balloon to permit monitoring of pressure during injection upstream of the balloon; or
wherein the catheter has a length in a range of 60 cm to 225 cm; or
wherein the catheter system is designed to facilitate both adult and pediatric endoscopic retrograde cholangiopancreatography (ERCP) procedures; or
wherein the first lumen has an opening proximate to the balloon; or
wherein the distal tip has a reduction in the caliber of the catheter in the distal quarter or third to increase flexibility; or
wherein the distal tip is comprised of materials different from the remainder of the catheter body to increase flexibility; or
wherein the balloon is spherical, cylindrical, olive, or pear shaped; or
wherein the balloon is comprised of durable materials that permit occlusion of the duct wall and minimizing friction within the ductal wall during maneuvering; or
wherein the balloon is comprised of silicone.

4-51. (canceled)

52. A method of performing endoscopic-mediated gene and protein delivery into liver, pancreas, and kidney organs, the method comprising the use of the catheter system of claim 1.

53. A method of nucleic acid or protein injection, the method comprising injecting contrast through the third lumen of the catheter system of claim 1, the third lumen being forward-facing.

54. The method of claim 53, wherein the method avoids biliary wall stress from lateral injection.

55. A method of hydrodynamic injection of fluid in a proximal catheter direction, the method comprising operating the catheter system of claim 1, the catheter having a third lumen opening proximate to the balloon.

56. A method of using the catheter system of claim 1, the method comprising inserting a guidewire having a maximum of 0.018, 0.025, or 0.035 inches in diameter into the second or third lumen.

57. A method of sealing pressure in an unused lumen during injection, the method comprising placing a cap or closed syringe on a connector of the unused lumen during injection to create a pressure seal and prevent fluid from escaping of the unused lumen of the catheter system of claim 1.

58. A method of introducing nucleic acid or protein solutions into a vessel or duct, the method comprising using the catheter system of claim 1, the vessel or duct selected from: bile duct, gallbladder, pancreatic duct, urethra, urinary bladder, ureter, renal pelvis, lung airways, or vascular system.

59. A method of treat genetic, neoplastic, autoimmune, ischemic, metabolic, or inflammatory changes in the human body, the method comprising using the catheter system of claim 1 to administer of nucleic acids or proteins at high fluid pressures.

60. A method of targeting an organ for endoscopic procedures, the method comprising using the catheter system of claim 1, the organ being of the gastrointestinal system, pulmonary system, or uretero-bladder system.

61. A method of treating liver, bile duct, pancreas, kidney, lung, heart or muscle diseases, the method comprising using the catheter system of claim 1.

62. A method of using the catheter system of claim 1, wherein the balloon is inflated to a predetermined size based on the caliber of a target vessel of duct

63. The method of claim 62, wherein the caliber of the vessel or duct is measured prior to use of the catheter system by an imaging modality.

64. The method of claim 62, wherein the caliber of the vessel or duct is measured fluoroscopically during use of the catheter system.

65. A method of using the catheter system of claim 1, wherein the balloon is not inflated to a predetermined size but is inflated based on the pressure within the balloon to ensure an adequate seal without being excessive; or
further comprising performing endoscopic-mediated gene and protein delivery into liver, pancreas, and kidney organs.

66. A catheter system comprising:
a catheter having a catheter body with a plurality of lumens extending through the catheter body including:
(a) a first lumen to partially deploy a fully covered self-expandable metallic stent;
(b) a second lumen for receiving a guidewire;
(c) a third lumen for receiving and transmitting a solution; and
(d) a fourth lumen for receiving a pressure catheter/transducer.

67. The catheter system of claim 66, wherein the second lumen and the third lumen are combined into a single lumen, wherein the guidewire is removed prior to injection; or
wherein the first lumen houses the stent and also allows injection of a gene therapy agent: or
wherein the partially deployed covered metallic stent is configured to remain attached to the catheter, allowing for ease of resheathing and removal from a target vessel or duct; or
wherein the partially deployed covered metallic stent comprises nitinol and is covered with silicone or polytetrafluorethylene (PTFE) membranes; or
wherein, after being deployed, the partially deployed covered metallic stent has a larger diameter than the target vessel or duct; or
wherein the partially deployed covered metallic stent has an olive-shaped distal tip; or
wherein the partially deployed covered metallic stent has an orifice at its distal tip configure to permit the passage of a guidewire; or
wherein the orifice is further configured to permit injection of the solution after the guidewire is removed; or
wherein the olive-shaped distal tip is configured to be retracted after partial stent deployment to the tip of the catheter such that the olive-shaped distal tip resides in the stent to block the distal end of the catheter during injection to prevent backflow; or
wherein the second lumen, the third lumen, and the fourth lumen are all housed in the olive-shaped distal tip; or
wherein the partially deployed covered metallic stent is configured to remain attached to the catheter, allowing for ease of resheathing and removal from a target vessel or duct.

68-78. (canceled)
